(19) United States
(12) Patent Application Publication
(10) Pub. No.: US 2024/0252558 A1
(43) Pub. Date: Aug. 1, 2024

(54) PROBIOTICS TO RESCUE MATERNAL IMMUNE ACTIVATION-INDUCED NEURODEVELOPMENT DEFICITS

(71) Applicant: The University of Chicago, Chicago, IL (US)

(72) Inventors: Erika C. Claud, Chicago, IL (US); Jing Lu, Chicago, IL (US); Lei Lu, Chicago, IL (US)

(21) Appl. No.: 18/425,731

(22) Filed: Jan. 29, 2024

Related U.S. Application Data
(60) Provisional application No. 63/461,375, filed on Apr. 24, 2023.
(60) Provisional application No. 63/482,048, filed on Jan. 29, 2023.

(51) Int. Cl. A61K 35/747; A61K 35/00; A61P 25/28

(52) U.S. Cl. CPC A61K 35/747; A61P 25/28; A61K 2035/115

(57) ABSTRACT

Provided herein are probiotics and methods of use thereof for treating one or more neurodevelopmental deficits in a subject resulting from exposure of the subject to maternal immune activation.

### PRIORITY STATEMENT

This application claims priority to U.S. Provisional Application No. 63/482,048, filed Jan. 29, 2023, and to U.S. Provisional Application No. 63/461,375, filed Apr. 24, 2023, the entire contents of each of which are incorporated herein by reference for all purposes.

### STATEMENT REGARDING FEDERAL FUNDING

This invention was made with government support under HD105234 and NS121432 awarded by the National Institutes of Health. The government has certain rights in the invention.

### FIELD

Provided herein are probiotics and methods of use thereof for treating one or more neurodevelopmental deficits in a subject resulting from exposure of the subject to maternal immune activation.

### BACKGROUND

Maternal acute and chronic inflammatory states derived from obesity, asthma, autoimmune disorders, depression, pre-eclampsia, and gestational diabetes; as well as environmental risks such as psychosocial stress, low socioeconomic status, exposure to smoking and pollution, and microbial dysbiosis are linked to diverse adverse neurodevelopmental outcomes in children. In particular, maternal inflammation in late-gestation poses increased risk for neurodevelopmental deficits in the offspring. Chorioamnionitis, characterized as bacterial infection resulting in acute inflammation of the placenta and/or fetal membranes, affects up to 10% of pregnancies and is highly associated with adverse neurodevelopmental outcomes namely periventricular leukomalacia, neonatal encephalopathy, and cerebral palsy. Accordingly, methods for diminish maternal immune activation-induced impairment of neurodevelopment are needed.

### SUMMARY

In some aspects, provided herein are methods of treating one or more neurodevelopmental deficits in a subject. In some embodiments, methods of treating one or more neurodevelopmental deficits in a subject comprise providing one or more probiotics to the subject via indirect maternal administration. In some embodiments, the one or more probiotics comprise Limosilactobacillus reuteri. In some embodiments, the subject was exposed to maternal immune activation (MIA) in utero. In some embodiments, the subject as exposed to MIA after at least 20 weeks of gestation. In some embodiments, the subject was exposed to MIA after at least 27 weeks of gestation. In some embodiments, the one or more neurodevelopmental deficits are selected from blood brain barrier (BBB) dysfunction, neuronal apoptosis, hypomyelination of white matter, neuroinflammation, microgliosis, astrogliosis, periventricular leukomalacia, neonatal encephalopathy, cerebral palsy, neonatal stroke, cognitive impairment, language impairment, autism, and schizophrenia. In some embodiments, the subject was exposed to MIA as a result of an autoimmune condition, asthma, an allergic condition, a viral infection, and/or a bacterial infection in the mother during gestation of the subject. In some embodiments, the subject was exposed to MIA as a result of chorioamnionitis during gestation of the subject. In some embodiments, the one or more probiotics are provided to the subject via indirect maternal administration during gestation of the subject. In some embodiments, the one or more probiotics are provided to the subject via indirect maternal administration after the subject is born (e.g. through breastmilk). In some embodiments, the one or more probiotics are provided to the subject via indirect maternal administration during gestation of the subject and via indirect maternal administration after the subject is born.

In some aspects, provided herein are methods of treating one or more neurodevelopmental deficits in a subject exposed to maternal immune activation (MIA) in utero, comprising providing one or more probiotics to the subject via indirect maternal administration. In some embodiments, the one or more probiotics comprise Limosilactobacillus reuteri. In some embodiments, the subject was exposed to MIA after at least 20 weeks of gestation. In some embodiments, the subject was exposed to MIA after at least 27 weeks of gestation. In some embodiments, the one or more neurodevelopmental deficits are selected from blood brain barrier (BBB) dysfunction, neuronal apoptosis, hypomyelination of white matter, neuroinflammation, microgliosis, astrogliosis, periventricular leukomalacia, neonatal encephalopathy, cerebral palsy, neonatal stroke, cognitive impairment, language impairment, autism, and schizophrenia. In some embodiments, the subject was exposed to MIA as a result of an autoimmune condition, asthma, an allergic condition, a viral infection, and/or a bacterial infection in the mother during gestation of the subject. In some embodiments, the subject was exposed to MIA as a result of chorioamnionitis during gestation of the subject. In some embodiments, the one or more probiotics are provided to the subject via indirect maternal administration during gestation of the subject. In some embodiments, the one or more probiotics are provided to the subject via indirect maternal administration after the subject is born (e.g. through breastmilk). In some embodiments, the one or more probiotics are provided to the subject via indirect maternal administration during gestation of the subject and via indirect maternal administration after the subject is born.

In some aspects, provided herein is a composition comprising one or more probiotics for use in a method of treating one or more neurodevelopmental deficits in a subject, the method comprising providing the composition to a subject via indirect maternal administration. In some embodiments, the one or more probiotics comprise Limosilactobacillus reuteri. In some embodiments, the subject was exposed to maternal immune activation (MIA) in utero. In some embodiments, the subject was exposed to MIA after at least 20 weeks of gestation. In some embodiments, the subject was exposed to MIA after at least 27 weeks of gestation. In some embodiments, the one or more neurodevelopmental deficits are selected from blood brain barrier (BBB) dysfunction, neuronal apoptosis, hypomyelination of white matter, neuroinflammation, microgliosis, astrogliosis, periventricular leukomalacia, neonatal encephalopathy, cerebral palsy, neonatal stroke, cognitive impairment, language impairment, autism, and schizophrenia. In some embodiments, the subject was exposed to MIA as a result of an autoimmune condition, asthma, an allergic condition, a viral infection, and/or a bacterial infection in the mother during gestation of the subject. In some embodiments, the subject was exposed to MIA as a result of chorioamnionitis during gestation of the subject. In some embodiments, the one or more probiotics are provided to the subject via indirect maternal administration during gestation of the subject. In some embodiments, the one or more probiotics are provided to the subject via indirect maternal administration after the subject is born (e.g. through breastmilk). In some embodiments, the one or more probiotics are provided to the subject via indirect maternal administration during gestation of the subject and via indirect maternal administration after the subject is born.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show Maternal L. reuteri supplementation during lactation rescued spatial learning deficit induced by maternal LPS exposure. (FIG. 1A) Significant difference during training at 12 weeks was found among SPF (n=26), LPS (n=11), L. reuteri (n=8) and LPS/L. reuteri (n=7) groups. SPF, L. reuteri and LPS/L. reuteri mice had significantly higher learning curve slopes than LPS by repeated measurement ANOVA. At training days 3 and 4, LPS mice took significantly more time to locate the escape platform than the mice of the other three treatment groups. Asterisks indicate significant differences of p-value at least <0.05. (FIG. 1B) Time in the platform quadrant during the probe trial was not different among the four treatment groups.

FIGS. 2A-2G show L. reuteri supplementation starting at birth reversed gestational LPS-induced vascular development deficits and hyperpermeability of the BBB in the offspring. Treatment did not affect (FIG. 2A) body weights or (FIG. 2B) total brain volume. (FIG. 2C) Maternal LPS significantly decreased the brain vascular volume compared to the saline control group and L. reuteri supplementation during lactation significantly minimized the LPS-induced vascular volume deficit (n=5,8,7,6, respectively). Bars with [symbol] denote significant difference between experimental groups (****p<0.0001, one-way ANOVA). (FIG. 2D) Baseline T1 values (seconds) were not different among the treatment groups. (FIG. 2E) Maternal LPS significantly increased the BBB permeability compared to the saline control offspring group and L. reuteri supplementation during lactation significantly minimized the LPS-induced BBB hyperpermeability. Quantification of permeability was derived from baseline T1 and post contrast (gd) T1 values. Permeability is presented as ΔT1 (baseline T1-post contrast T1)/vessel volume. Bars with [symbol] denote significant difference between experimental groups (****p<0.0001, one-way ANOVA). (FIG. 2F) Representative T₂W and T1 images of brains. Panels represent treatment groups (A) SPF, (B) LPS, (C) L. reuteri, and (D) LPS/L. reuteri. 1st row (gray)—Three middle slices of T₂W brain images; 2^{nd} row—measured mouse brain T1 maps before contrast agent injection; 3^{rd} row—measured mouse brain T1 maps 25 minutes after contrast agent injection. The color bar underneath the maps shows scales (value) of the T1 map. (FIG. 2G) Representative images of mouse brain blood vessels (red color) obtained from the TOF datasets superimposed over T₂W images (gray). Panels represent treatment groups (A) SPF, (B) LPS, (C) L. reuteri, and (D) LPS/L. reuteri. For visual inspection, Maximum Intensity Projection (MIP) image shown in the right column was generated from TOF datasets. The MIP connects the high intensity dots of the blood vessels in three dimensions.

FIGS. 3A-3G show astrogliosis in two-week old offspring induced by maternal LPS was reduced by maternal L. reuteri supplementation. FIGS. 3A-3D show representative images of fluorescence microscopy of claudin-5 (location of the brain capillaries, red), GFAP astrocyte (green), and DAPI (nuclei, blue). Seven to ten sections per mouse of three mice were inspected in each group. Stronger than control SPF GFAP staining (FIG. 3A) was observed around the blood vessel and in the brain with maternal LPS insult (FIG. 3B). Maternal supplemented of L. reuteri (FIG. 3C) without or (FIG. 3D) with maternal LPS had GFAP levels similar to the control group. Based on quantification of astrocyte activation using ImageJ (NIH), (FIG. 3E) Overall expression of claudin-5 was not affected by treatment. (FIG. 3F) GFAP expression in the vicinity of the blood vessel and (FIG. 3G) GFAP expression in the brain were expressed as GFAP integral density (IntDen) levels over claudin-5 levels. Bars with [symbol] denote significant difference between experimental groups (all n=3, at least p<0.05).

FIGS. 4A-4D show relative abundance of bacterial communities among the treatment groups. Relative abundance at phylum level of two (FIG. 4A) and 12 (FIG. 4B) weeks old fecal samples. Relative abundance at family level of two (FIG. 4C) and 12 (FIG. 4D) weeks old fecal samples (all n=5).

FIGS. 5A-5C show α-diversity and Bray-Curtis principal component analysis of fecal microbiota. α-diversity metrics of (FIG. 5A) observed, chao1, and Shannon diversity of two- and 12-weeks old mouse fecal samples calculated using R package. No significant difference was found among the treatment groups in any of the metrics. Principal component analysis (PCoA) scores are plotted based on the relative abundance of fecal microbiota at the genus level of (FIG. 5B) two and (FIG. 5C) 12 weeks old mouse fecal samples. The percentage of variation explained by the principal component is indicated on the axis. A. SPF B. LPS C. Reuteri D. LPS/Reuteri. Significant separation in the gut microbiome composition (β-diversity) was observed among different treatment groups (all n=5) by PERMANOVA (p=0.001).

FIGS. 6A-6D show principal component analysis and heatmap of serum and brain metabolite profiles at two weeks of age. Principal component analysis (PCoA) scores are plotted based on the normalized peak area of (FIG. 6A) serum and (FIG. 6B) brain metabolites of two weeks old mice. A Hierarchical clustering was applied to arrange the metabolites based on the similarity of the abundance among samples. For two weeks old samples, (FIG. 6C) 77 significantly different serum features and (FIG. 6D) 88 significantly different brain features were plotted (One-way ANOVA test with Benjamini-Hochberg method-adjusted p value<0.05, all n=3).

FIGS. 7A-7D show principal component analysis and heatmap of serum and brain metabolite profiles at 12 weeks of age. Principal component analysis (PCoA) scores are plotted based on the normalized peak area of (FIG. 7A) serum and (FIG. 7B) brain metabolites of 12 weeks old mice. For 12 weeks old samples, top 100 features by ANOVA were plotted for (FIG. 7C) serum and (FIG. 7D) brain samples with * indicating a significant difference among the four treatment groups (One-way ANOVA test with Benjamini-Hochberg method-adjusted p value<0.05, all n=4)

FIGS. 8A-8G show significantly different metabolic features between the serum and brain pool. Features were presented using their putatively identified names. (FIG. 8A) Venn diagram showing the significantly different metabolic features between the serum and brain pool. The number at the intersection represents the number of significantly different metabolites shared by serum and brain, while the number out of the intersection represents the number of unique metabolites in each pool. (FIG. 8B) Of the shared metabolites in both pools, 1-palmitoyl-phosphatidylcholine in the brain of the LPS group was significantly less than that of the SPF group (p=0.0016). 1-palmitoyl-phosphatidylcholine levels in both Reuteri and LPS/Reuteri groups were significantly higher than that of the LPS group (p=0.048 and p<0.0001, respectively). (FIG. 8C) In the unique brain pool, both Lyso PC (20:5) and palmitolycarnitine in the brain of LPS group were significantly less than that of the SPF group (p=0.0003 and p=0.0197, respectively). These two metabolite levels in both Reuteri and LPS/Reuteri groups were significantly higher than that of the LPS groups (for Lyso PC (20:5), p=0.045 and p=0.0009, respectively; for palmitolycarnitine, p=0.0079 and p=0.0009, respectively). (FIG. 8D) In the unique serum pool, 1-(1Z-Hexadecenyl)-sn-glycero-3-phosphocholine level in the LPS group was significantly less (p=0.016) and PC(P-18:0/22:6) (p=0.039) level was significantly higher than that of the SPF group. These two metabolite levels in both the Reuteri and LPS/Reuteri groups were similar to that of the SPF group. All data were analyzed by one-way ANOVA with Tukey's post hoc test, all n=3. Unique features using their putatively identified names that crossed the BBB of the offspring upon maternal LPS challenge were identified in (FIG. 8E) Venn diagram, revealing that there were two features that uniquely crossed the BBB under the influence of LPS. (FIG. 8F) The levels of 8-HETE and (FIG. 8C) 2-arachidonoyl-lysophosphatidylcholine were not different among the four treatment groups (one-way ANOVA). Unique features that crossed the BBB of the offspring upon maternal L. reuteri exposure during lactation were identified in (FIG. 8G) Venn diagram, revealing that there were 14 unique features that crossed the BBB under the influence of L. reuteri.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a metabolite” is a reference to one or more metabolites and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., primates, dogs, cats, cows, horses, sheep, poultry, fish, crustaceans, etc.). In some embodiments, the subject is a human. In some embodiments, the subject is breastfeeding (e.g. nursing). In some embodiments, the subject is an infant. The term “infant” refers to a subject less than 2 years of age. In some embodiments, the subject is an infant subject that has already been born. In some embodiments, the subject is still in utero (e.g. a fetus).

### DETAILED DESCRIPTION

In some aspects, provided herein are methods. In some aspects, provided herein are methods of treating one or more neurodevelopmental deficits in a subject. In some embodiments, the subject was exposed to maternal immune activation (MIA) in utero. In some embodiments, the one or more neurodevelopmental deficits result from or likely result from the exposure to MIA. In some aspects, provided herein are methods of treating one or more neurodevelopmental deficits in a subject exposed to maternal immune activation (MIA) in utero.

In some embodiments, the methods described herein comprise providing one or more probiotics to the subject via indirect maternal administration. As used herein, the term “indirect maternal administration” indicates that an agent (e.g. a probiotic) is provided to the subject indirectly through the maternal figure, as opposed to being delivered directly to the subject. As used herein, the term “maternal figure” and “mother” are used interchangeably. In some embodiments, the term “mother” refers to the biological mother of the subject, and the subject was exposed to MIA due to MIA occurring in the biological mother during gestation. However, the term “mother” when used in reference to “indirect maternal administration” does not necessarily indicate the biological mother of the subject. For instance, the “maternal figure” or “mother” through which indirect maternal administration is accomplished may refer to a person who is breastfeeding the subject, but that person may not necessarily be the biological mother of the subject. For example, in some embodiments “indirect maternal administration” indicates that an agent (e.g. a probiotic) is provided to the subject indirectly through the maternal figure, such as by passing from breastmilk from the maternal figure to the infant or passing through the placenta from the mother to the infant in utero.

In some embodiments a probiotic is provided to the maternal figure (e.g. orally), and then provided to the subject via indirect maternal administration by passing from the breastmilk of the maternal figure to the subject. In such embodiments, the probiotic is provided to the subject by indirect maternal administration (e.g. passing through the breastmilk) after birth. Accordingly, in some embodiments the methods described herein (e.g. methods of treating one or more neurodevelopmental deficits in a subject) comprise providing one or more probiotics to the subject via indirect maternal administration, wherein the probiotics are provided to the subject after birth.

As another example of indirect maternal administration, in some embodiments a probiotic is provided to the mother (e.g. orally), and then passes from the mother to the subject (e.g. through the placenta) during gestation (e.g. in utero). In such embodiments, the probiotic is provided to the subject by indirect maternal administration during gestation. Accordingly, in some embodiments the methods described herein (e.g. methods of treating one or more neurodevelopmental deficits in a subject) comprise providing one or more probiotics to the subject via indirect maternal administration, wherein the probiotics are provided to the subject during gestation (e.g. in utero, by passing through the placenta).

In some embodiments, the one or more probiotics are provided to the subject through indirect maternal administration both in utero and following birth. For example, in some embodiments the one or more probiotics are provided to the subject through indirect maternal administration in utero (e.g. during gestation, passing through the placenta), and following birth via indirect maternal administration (e.g. through breastmilk).

In some embodiments, the one or more probiotics comprise Limosilactobacillus reuteri. In some embodiments, the one or more probiotics comprise a Bifidobacterium species (e.g. Bifidobacterium longum, Bifidobacterium breve, Bifidobacterium animalis, Bifidobacterium bifidum, etc.).

In some embodiments, the one or more probiotics are provided to the subject in multiple doses occurring over a given window of time. For example, in some embodiments the one or more probiotics are provided to the subject via multiple doses of indirect maternal administration occurring over a given window of time. For example, in some embodiments a pregnant mother receives multiple doses of one or more probiotics (e.g. orally) over a given window of time, and the probiotic is thus provided to the subject (e.g. the fetus) by passing through the placenta. As another example, in some embodiments a nursing maternal figure receives multiple doses of one or more probiotics over a given window of time, and the probiotic is thus provided to the subject (e.g. the nursing infant) by passing from the maternal figure to the subject in breastmilk. In some embodiments, the window of administration of the one or more probiotics (e.g. the window of time that the pregnant mother or the nursing mother receives the one or more probiotics, and thus the window of time that the subject is provided the one or more probiotics via indirect maternal administration) is at least one day, at least one week, at least two weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 4 months, at least 5 months, or at least 6 months. In some embodiment, indirect maternal administration occurs at least once daily over the given window of time. In some embodiments, indirect maternal administration occurs at least twice per day. In some embodiments, indirect maternal administration occurs daily. In some embodiments, indirect maternal administration occurs every other day.

Generally speaking, suitable doses of probiotics received by (e.g. consumed by) the mother (and thus provided to the subject via indirect maternal administration) are given as a number of colony forming units (CFU) of the probiotic bacteria. In some embodiments, the mother receives the one or more probiotics in multiple doses over a given window of time, as described above, wherein each of the one doses is about 10 million CFU to about 50 billion CFU. For example, in some embodiments each of the one or more doses is about 10 million to about 50 billion CFU, about 100 million to about 45 billion CFU, about 200 million to about 40 billion CFU, about 300 million to about 35 billion CFU, about 400 million to about 30 billion CFU, about 500 million to about 25 billion CFU, about 600 million to about 20 billion CFU, about 700 million to about 15 billion, about 800 million to about 10 billion, about 900 million to about 1 billion, or about 1 billion CFU. In some embodiments, each of the one or more doses is about 10 million CFU, about 50 million CFU, about 100 million CFU, about 200 million CFU, about 300 million CFU, about 400 million CFU, about 500 million CFU, about 600 million CFU, about 700 million CFU, about 800 million CFU, about 900 million CFU, about 1 billion CFU, about 2 billion CFU, about 3 billion CFU, about 4 billion CFU, about 5 billion CFU, about 6 billion CFU, about 7 billion CFU, about 8 billion CFU, about 9 billion CFU, about 10 billion CFU, about 11 billion CFU, about 12 billion CFU, about 13 billion CFU, about 14 billion CFU, about 15 billion CFU, about 16 billion CFU, about 17 billion CFU, about 18 billion CFU, about 19 billion CFU, or about 20 billion CFU.

In some embodiments, the subject was exposed to maternal immune activation (MIA) in utero. As used herein, the term “maternal immune activation” or “MIA” refers to an increase in the levels of inflammatory molecules in a pregnant woman (e.g. a mother). In some embodiments, MIA refers to activation of inflammatory pathways in the mother resulting in increased levels of cytokines and chemokines. In some embodiments, increased levels of inflammatory cytokines and chemokines in the mother cross the placental barrier and/or the blood brain barrier, resulting in altered fetal neural development. In some embodiments, the subject was exposed to MIA after at least 20 weeks of gestation. In some embodiments, the subject was exposed to MIA after at least 27 weeks of gestation. For example, in some embodiments the subject was exposed to MIA after at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, or at least 40 weeks of gestation.

Numerous maternal conditions may cause maternal immune activation, including autoimmune conditions, asthma, allergic conditions, viral infections, and bacterial infections. In some embodiments, the subject was exposed to MIA as a result of an autoimmune condition in the mother. In some embodiments, the subject was exposed to MIA as a result of asthma or an allergic condition in the mother. In some embodiments, the subject was exposed to MIA as a result of a bacterial infection in the mother. In some embodiments, the bacterial infection occurred after at least 20 weeks of pregnancy (e.g. after at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, or at least 40 weeks of pregnancy). For example, in some embodiments the subject was exposed to MIA as a result of chorioamnionitis (e.g. an infection of the placenta and the amniotic fluid, also referred to as intraamniotic infection). In some embodiments, the subject was exposed to MIA as a result of viral infection in the mother. Exemplary viral infections in the mother that may expose the subject (e.g. the developing fetus) to MIA include, for example, varicella, cytomegalovirus, mumps, herpes simplex virus, and SARS-CoV-2. In some embodiments, the viral infection occurred after at least 20 weeks of pregnancy (e.g. after at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, or at least 40 weeks of pregnancy).

In some embodiments, the one or more neurodevelopmental deficits are caused by or likely caused by exposure to MIA in utero (e.g. during gestation). In some embodiments, the one or more neurodevelopmental deficits are selected from blood brain barrier (BBB) dysfunction, neuronal apoptosis, hypomyelination of white matter, neuroinflammation, microgliosis, astrogliosis, periventricular leukomalacia, neonatal encephalopathy, cerebral palsy, neonatal stroke, cognitive impairment, language impairment, autism, and schizophrenia. As used herein, the term “blood brain barrier dysfunction” or “BBB dysfunction” is used in the broadest sense and refers to any deficit or dysregulation of the blood brain barrier, including changes in permeability (e.g. hyperpermeability), transport mechanisms, structural integrity of cellular junctions, and the like.

“Treating” the one or more neurodevelopmental deficits in the subject is used in the broadest sense and refers to ameliorating the one or more neurodevelopmental deficits, reducing the severity of the one or more neurodevelopmental deficits, or preventing or reducing the likelihood of the subject developing the one or more neurodevelopmental deficits. In some embodiments, the subject is exposed to MIA in utero, and the method of treating the one or more neurodevelopmental deficits in the subject involves providing to the subject one or more probiotics via indirect maternal administration while the subject is still in utero (before birth, during gestation). In such embodiments, “treating” the one or more neurodevelopmental deficits may refer to preventing or reducing the likelihood of the subject developing one or more neurodevelopmental deficits as a result of the exposure to MIA in utero. In such embodiments, “treating” the one or more neurodevelopmental deficits may refer to ameliorating or reducing the severity of the one or more neurodevelopmental deficits resulting or likely resulting from the exposure to MIA in utero. In some embodiments, the subject is exposed to MIA in utero, and the method of treating the one or more neurodevelopmental deficits in the subject involves providing to the subject one or more probiotics via indirect maternal administration after birth, such as through breastmilk. In such embodiments, “treating” the one or more neurodevelopmental deficits may refer to ameliorating or reducing the severity of the one or more neurodevelopmental deficits resulting or likely resulting from the exposure to MIA in utero.

Administration of probiotics to the subject via indirect maternal administration (e.g. during gestation and/or through breastmilk after birth) may convey several beneficial properties to subject that contribute to the treatment of one or more neurodevelopment deficits in the subject (e.g. following exposure to MIA in utero). For example, in some embodiments administration of probiotics to the subject via indirect maternal administration prevents or significantly improves blood brain barrier hyperpermeability in the subject. As another example, in some embodiments administration of probiotics to the subject via indirect maternal administration reduces or prevents astrogliosis in the subject.

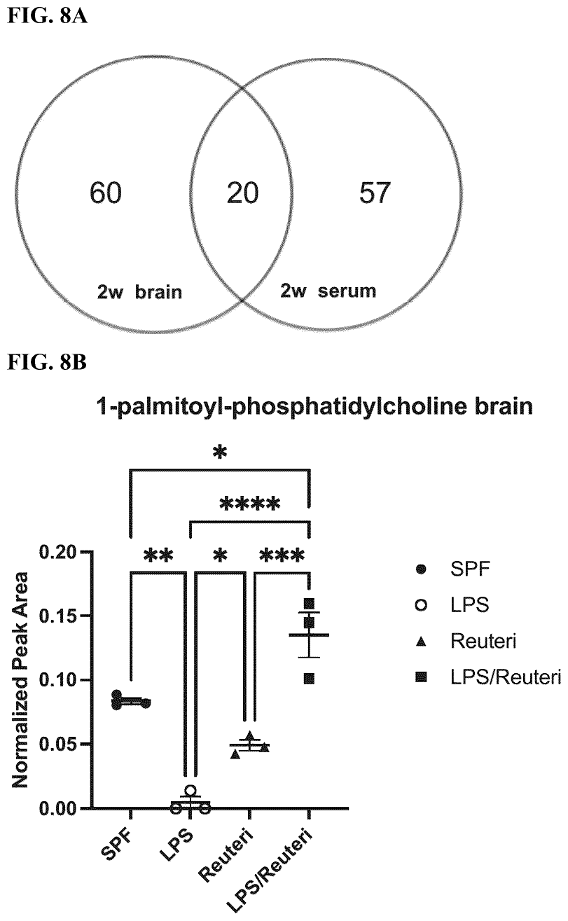

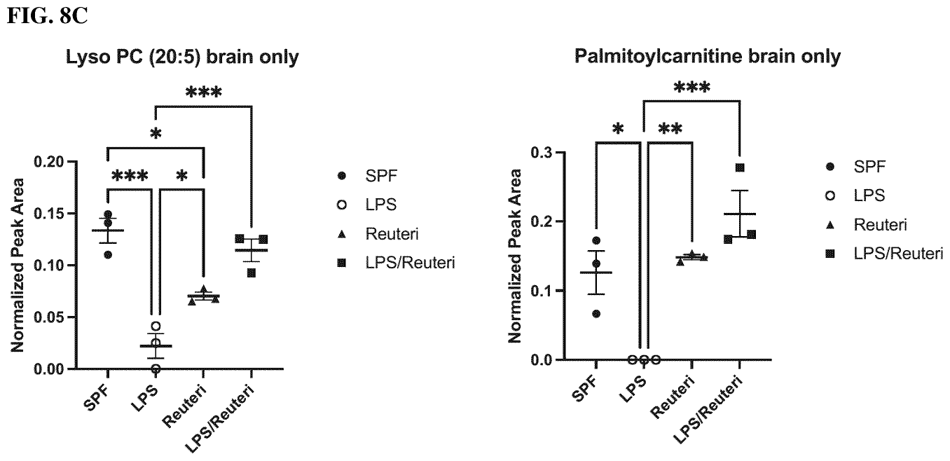

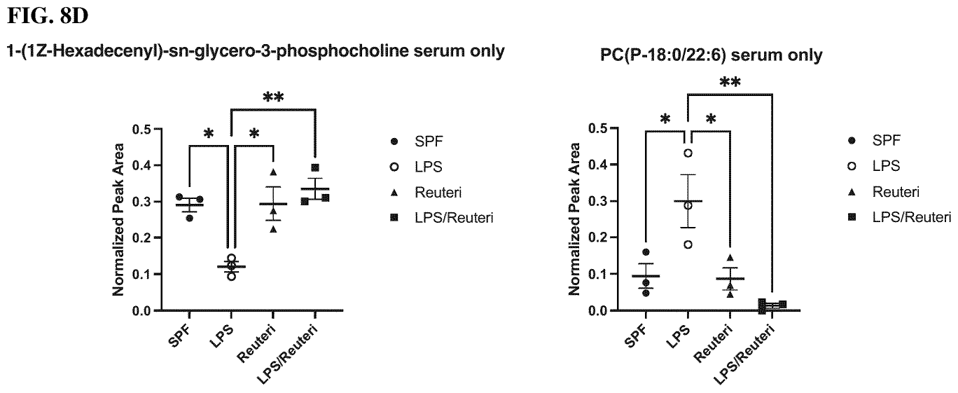

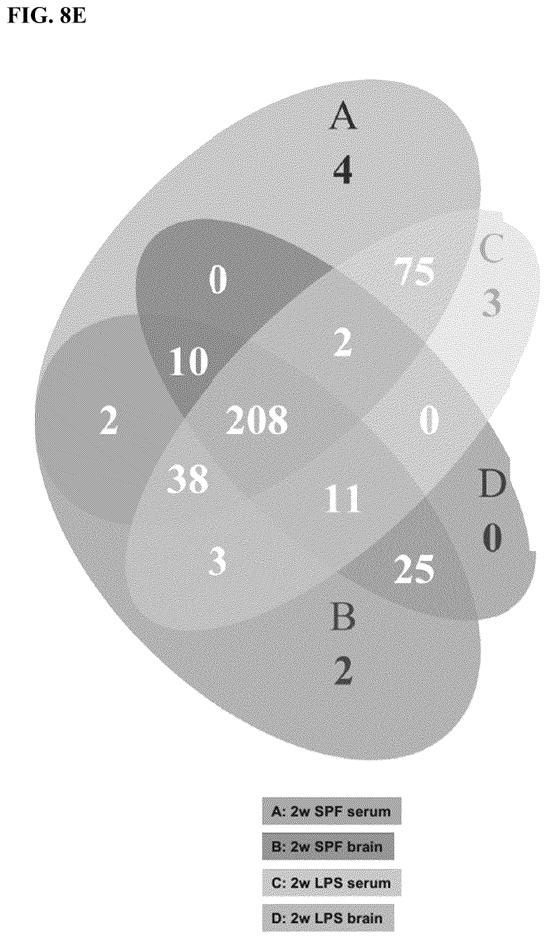

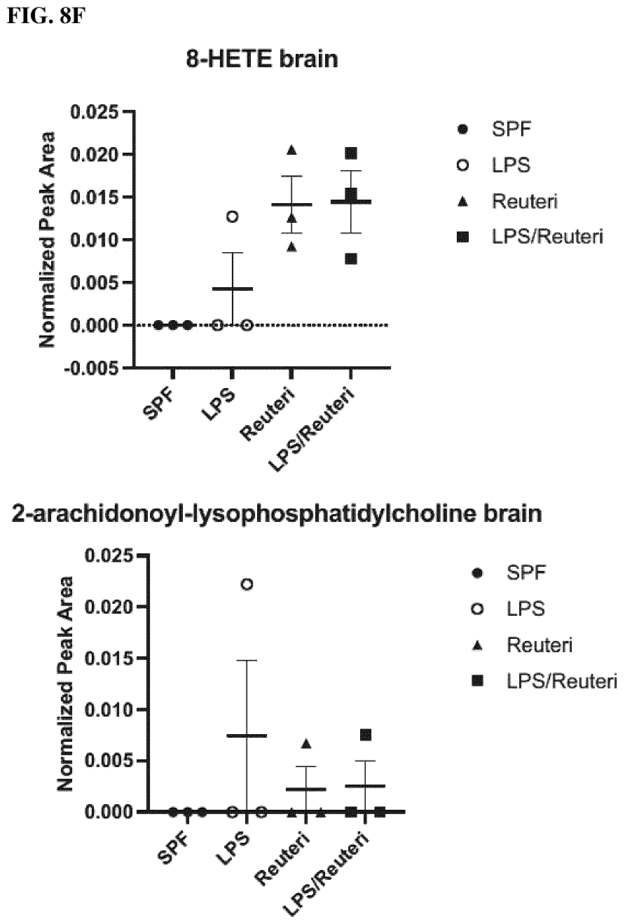

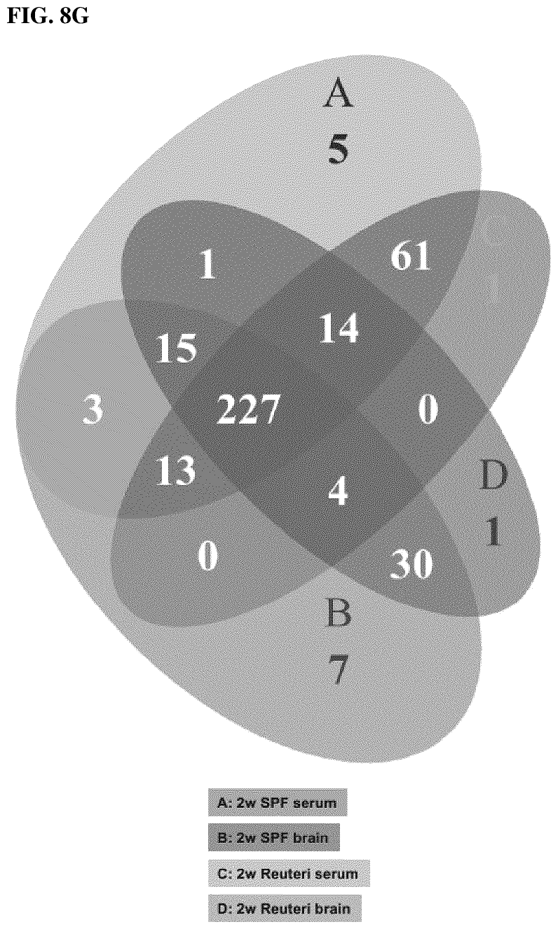

In some embodiments, administration of probiotics to the subject via indirect maternal administration causes beneficial changes in the microbiome or metabolome of the mother, which conveys beneficial properties to the subject that treat the one or more neurodevelopmental deficits in the subject. As used herein, the term “microbiome” refers to the microbes (e.g., bacteria, fungi, protists) their genetic elements (genomes) in a defined environment. The term “gut microbiome” refers to the microbes present in the digestive tract of a subject. As used herein, the term “metabolome” refers to the complete set of small-molecule metabolites (e.g., metabolic intermediates, hormones signaling molecules, secondary metabolites, etc.) found within a defined biological environment or sample. The term “gut metabolome” refers to the metabolites present in the digestive tract of a subject. In some embodiments administration of probiotics to the subject via indirect maternal administration modulates the β-diversity of the microbiome of mother and/or the subject. In some embodiments, administration of probiotics to the subject via indirect maternal administration modulates the metabolome of the mother and/or of the subject. In some embodiments, administration of probiotics to the mother modulates metabolite production in the mother, which metabolites are passed to the subject (e.g. via breastmilk, via the placenta in utero) and cross the blood brain barrier of the subject. Accordingly, the metabolites are able to enter the nervous system of the subject and exert beneficial effects. In some embodiments, administration of probiotics to the mother causes new metabolites to be produced in the mother (e.g. metabolites which are not present in the mother absent administration of the probiotics probiotics) and these new metabolites cross the blood brain barrier to convey beneficial effects to the subject. Exemplary metabolites are shown in FIG. 8. In some embodiments, indirect maternal administration of probiotics increases levels of one or more lysophosphatidylcholines, palmitoylcarnitines, and/or eicosanoids in the mother and/or in the subject.

### EXPERIMENTAL

### Example 1

Communication between the gut microbiota and the central nervous system (CNS) is known as the gut-microbiome-brain axis, and it is possible that brain development is influenced by early life microbiota. Probiotics are live microorganisms, which when administered in adequate amounts, confer a benefit for the host. Limosilactobacillus reuteri (formerly Lactobacillus reuteri, L. reuteri) is a probiotic that exhibits beneficial traits in gut physiology including production of antimicrobial molecules to prevent pathogen overgrowth, and regulation of bacterial colonization, mucosal barrier integrity, mucosal IgA responses and production of anti-inflammatory cytokines. However, even though the gut microbiota can be manipulated with probiotics, there are concerns about direct administration of probiotics to newborns due to the immature immune system of newborns, suspected higher susceptibility to infections, and sepsis.

The mechanisms by which gut microbiota communicate with the CNS are still largely unknown. Suggested potential links include pathways of systemic inflammation, immune surveillance, and production of metabolites/neuromodulators/neurotransmitters. A common trait of these pathways is the release of microbial mediators (i.e., cytokines, metabolites, activated immune cells) into the systemic system. Whether or what microbial mediators reach the CNS to influence brain function is dependent on the systemic communication between peripheral blood and the tightly regulated CNS barrier known as the blood-brain barrier (BBB). In addition, these microbial mediators might have a direct impact on BBB development and function, and thereby indirectly regulate CNS functions, without even entering the CNS. Dysfunctions of the BBB have been implicated in Parkinson's and Alzheimer's disease in adult populations as well as in several neurological disorders in children including cerebral palsy, neonatal stroke, and autism. Aberrant microbiota and microbiota-related systematic changes may disrupt BBB integrity. However, specific microbial mediators associated with BBB disruption under the influence of maternal inflammation and the resulting influence on offspring neurodevelopment have not been studied.

Some of the properties of the BBB in the developing brain are not yet fully mature at birth. Accordingly, herein it was investigated whether maternal probiotic administration alleviates the adverse impact of maternal immune activation (MIA) on offspring BBB development and function. Specifically, it was investigated whether L. reuteri exposure during lactation (days 1-21 of offspring life) would improve MIA-induced BBB dysfunction and neurodevelopment deficits through altering the metabolites that cross the BBB. Herein it is demonstrated that MIA, modeled by maternal LPS exposure, induces a development deficit of the vasculature of the BBB. This was associated with distinct shifts in the serum and brain metabolome of the offspring. Maternally administrated L. reuteri during lactation normalized MIA-induced BBB development deficits and increased permeability, promoted entry of specific bacterial metabolites to the brain, and significantly improved spatial learning later in life.

### Results

Maternal Administration of L. reuteri During Lactation Improved Spatial Learning in the Offspring after Maternal LPS Exposure

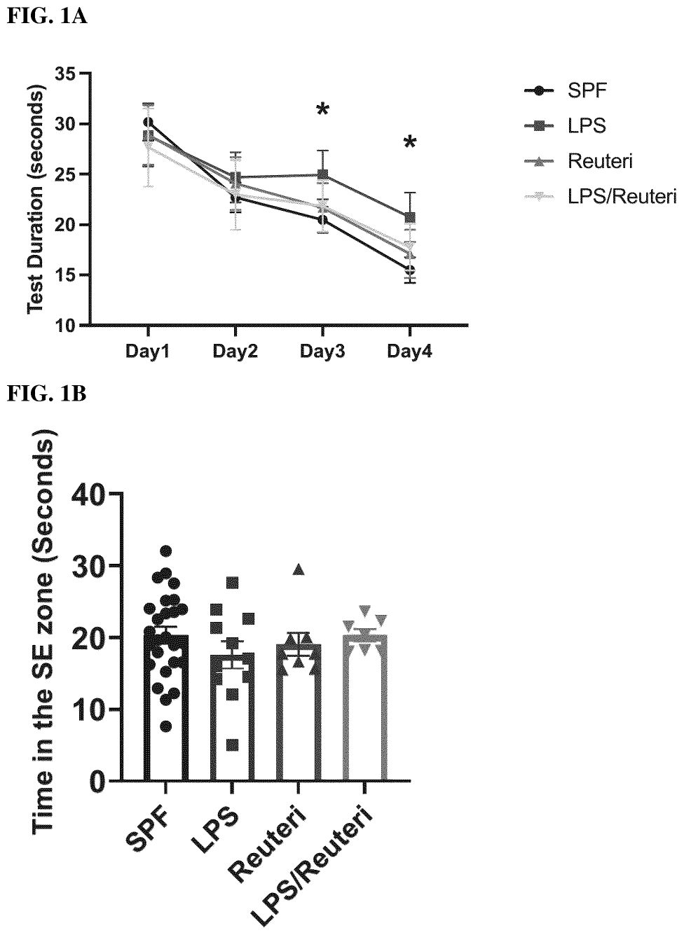

Maternal LPS exposure is an animal model of chorioamnionitis and infection associated MIA for the study of long-term outcomes in the offspring. To determine if the neurodevelopmental outcomes of the offspring from the maternal LPS exposure model could be improved by maternally administrated L. reuteri during lactation, Morris water maze was used to assess spatial learning and memory in the offspring at 12 weeks of age. Four testing groups were used SPF (n=26), LPS (n=11), Reuteri (n=8), and LPS/Reuteri (n=7). In calculating escape latency during testing days by two-way ANOVA with repeated measures (trials), both the main effect factors of testing day (F_{3,192}=176.8, p<0.0001) and treatment (F_{3,192}=14.68, p<0.0001) were significant across all groups, demonstrating that the latency time to find the hidden platform decreased during training in all, thus all groups were learning (FIG. 1a). However, offspring from the maternal LPS exposure group showed a longer latency to escape onto the hidden platform on the 3rd and 4th day when compared to control, Reuteri or LPS/Reuteri groups (two-way repeated measure ANOVA simple effect by Tukey's post hoc test. Adjusted (for multiple comparison) p values at day 3, LPS vs SPF p<0.0001, LPS vs Reuteri p=0.0089, LPS vs LPS/Reuteri p=0.0203; at day 4, LPS vs SPF p<0.0001, LPS vs Reuteri p=0.0024, LPS vs LPS/Reuteri p=0.0279). These data demonstrate that maternal LPS exposure impaired spatial learning ability when compared to controls, but that L. reuteri supplementation during lactation reversed the spatial learning impairment. In the probe trial, time spent in the quadrant where the hidden platform had been (FIG. 1b) did not differ among the four groups, indicating that spatial memory was not affected.

Maternal L. reuteri Supplementation During Lactation Restored Maternal LPS-Induced Impairment of Brain Vascular Development and BBB Hyperpermeability in the Offspring

To determine if the alteration in the offspring spatial learning associated with maternal LPS exposure was linked to early BBB dysfunction, overall brain vascular development was first evaluated. Rodents are only susceptible to inflammation-induced increased permeability of cerebral blood vessels before P20, a stage of brain development equivalent to 22-40 weeks of gestation in humans. Accordingly, in the current study mice at two weeks old were subjected to MRI Time of flight (TOF) to visualize flow within vessels, without the need to administer contrast.

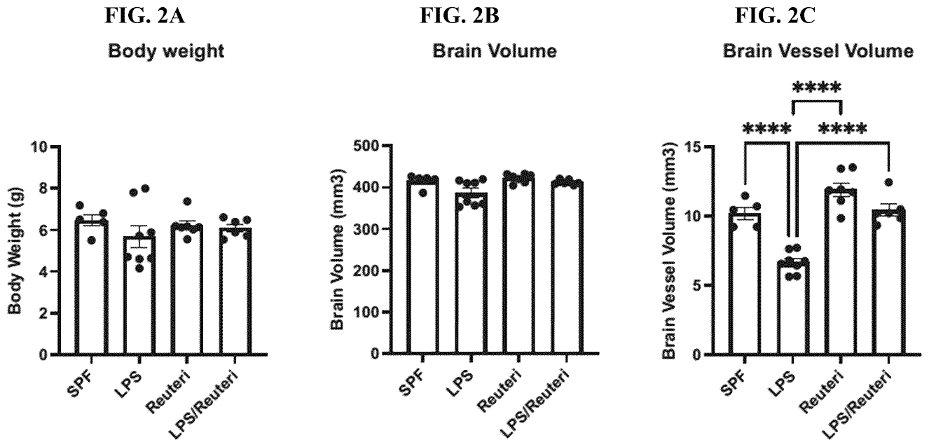

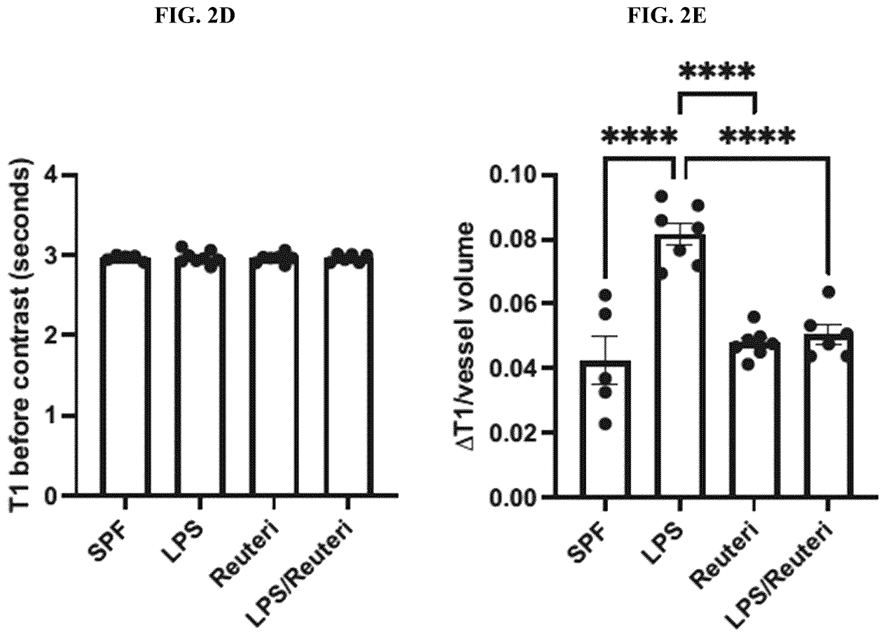

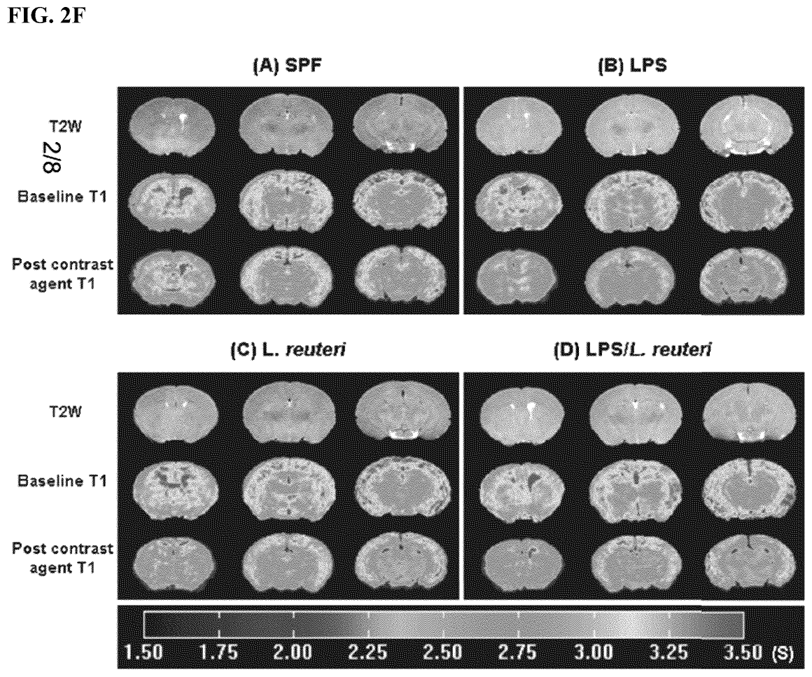

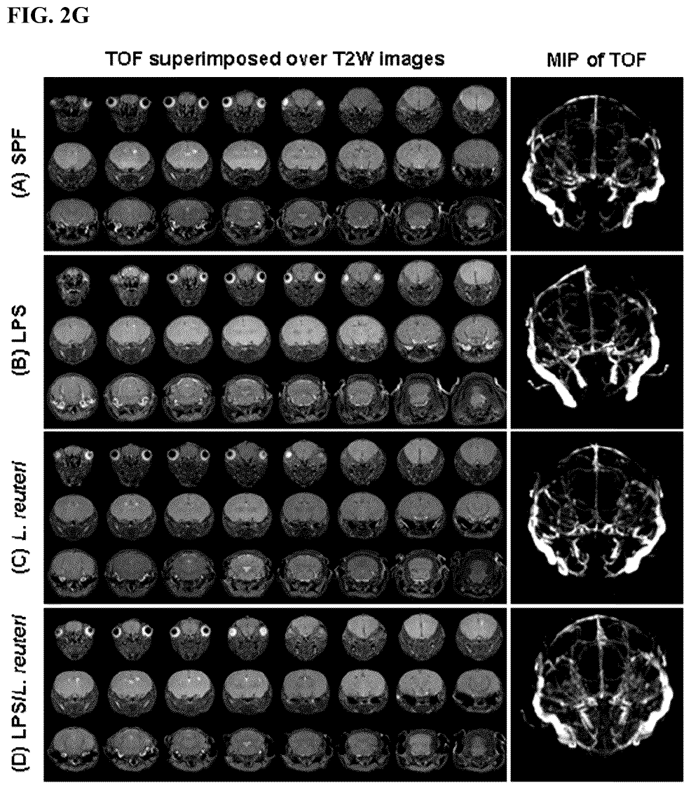

Mouse body weights at two weeks of age were not different (FIG. 2a, p>0.05, ANOVA) among the treatment groups. There was also no statistical difference in brain volume among the treatment groups (quantification in FIG. 2b, p>0.05, ANOVA) based on the T₂W imaging of brain anatomy (FIG. 2f top panel). However, prenatal exposure to LPS compared to saline SPF control group, did significantly decrease the total blood vessel volume based on TOF measurement (FIGS. 2g (A) and 2g (B), quantification in FIG. 2c, p<0.0001, Tukey's post hoc test after one-way ANOVA). L. reuteri supplementation alone during lactation did not affect the vascular volume (FIG. 2g (C)) but reestablished the vascular volume when compared to the maternal LPS challenged alone group (FIG. 2g (D), p<0.0001, Tukey's post hoc test after one-way ANOVA).

BBB permeability was evaluated by calculating ΔT1 (longitudinal relaxation time) value between the before contrast T1 value (baseline T1 in FIG. 2f middle panel) and post contrast agent T1 value (FIG. 2f bottom panel). Although there was no statistical difference between the average baseline T1 values (FIG. 2d, the amount of contrast in the brain was significantly higher in the maternal LPS challenged group when compared to the control group (FIG. 2e, p<0.0001, Tukey's post hoc test after one-way ANOVA), indicating higher BBB permeability in the maternal LPS exposed offspring at two-weeks of age. Remarkably, maternal L. reuteri administration during lactation repaired the prenatal LPS-induced BBB hyperpermeability (FIG. 2e, p<0.0001, Tukey's post hoc test after one-way ANOVA). These data demonstrate for the first time that gestational MIA-induced by LPS significantly impairs vascular development and permeability of the BBB in the offspring and that L. reuteri supplementation starting at birth can reverse these deficits.

Maternal Administration of L. reuteri During Lactation Ameliorated Astrogliosis in the Offspring after Maternal LPS Exposure

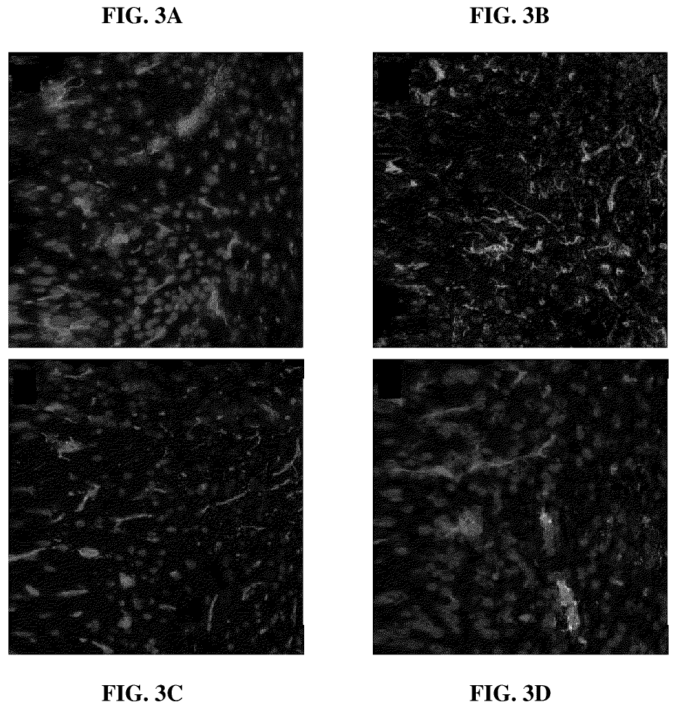

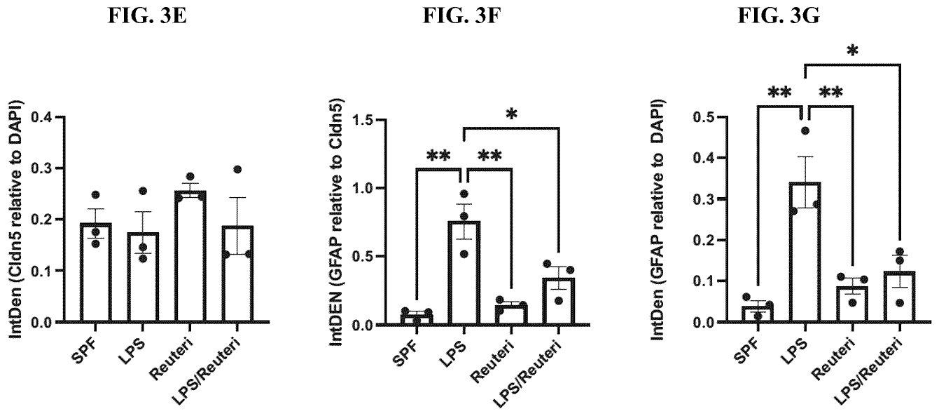

Astrogliosis is a common feature of astrocytes during BBB disruption, characterized by upregulation of the phenotypical astrocyte protein glial fibrillary acidic protein (GFAP) during CNS insults. To determine if maternal LPS-induced changes in BBB were linked to astrocyte activation, immunohistochemical staining for GFAP (FIG. 3, green) and the BBB-specific tight junction protein claudin-5 (FIG. 3, red) to define brain blood vessels was performed. No differences in claudin-5 protein levels (FIG. 3a, SPF; FIG. 3b, LPS; FIG. 3c, Reuteri; FIG. 3d, LPS/Reuteri) quantified by integrated intensity (IntDen) levels (using ImageJ (NIH)) in the cerebrum of the offspring from either maternal L. reuteri-supplemented or un-supplemented groups with or without prenatal LPS insult were found (FIG. 3e, p>0.05, ANOVA). Maternal LPS challenge did induce a significant elevation in GFAP staining (green) when compared to saline (FIG. 3a), both around the blood vessel (identified by claudin-5 staining, red) and in the cerebral tissue (FIG. 3b), with quantification presented as GFAP IntDen over claudin-5 IntDen in FIG. 3f (p=0.0012) and GFAP over DAPI IntDen (nuclei staining) in FIG. 3g (p=0.0088), respectively (Tukey's post hoc test after one-way ANOVA). Maternal L. reuteri exposure during lactation did not change the GFAP expression but significantly diminished maternal LPS-induced increased GFAP expression (FIGS. 3c and 3d, with quantification in FIGS. 3f p=0.0247, and 3g p=0.0381, respectively, Tukey's post hoc test after one-way ANOVA). These data demonstrate that BBB susceptibility to maternal-LPS-induced disruption is complemented by astrogliosis and that maternal L. reuteri during lactation can reduce astrogliosis at both a global brain level and specifically at the BBB.

Maternal Administration of L. reuteri During Lactation Altered Offspring Microbiome β-Diversity after Maternal LPS Exposure

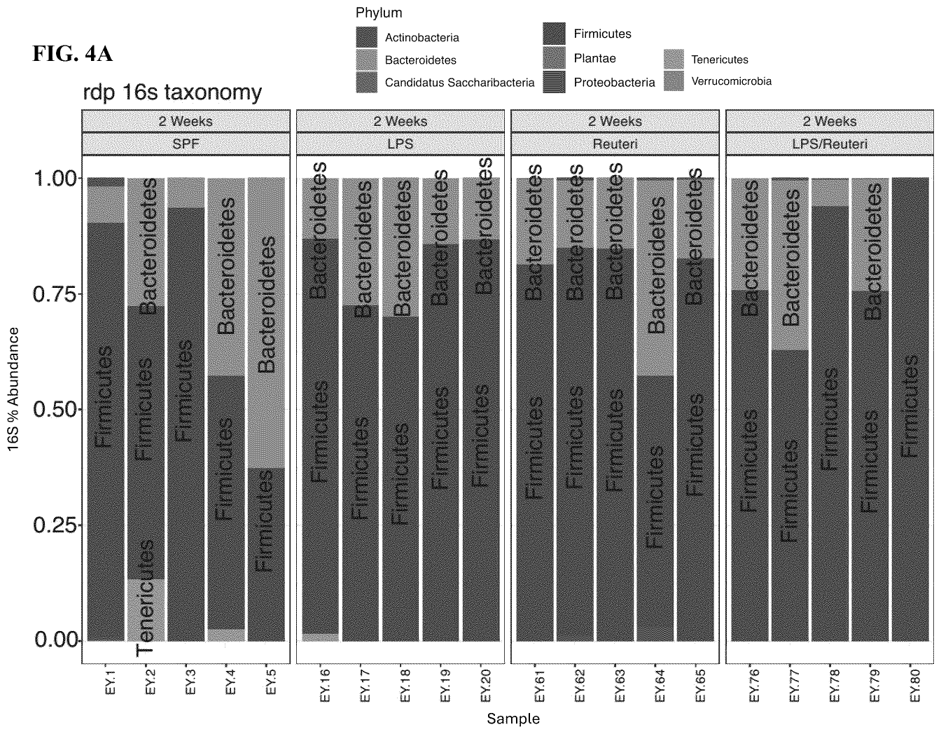

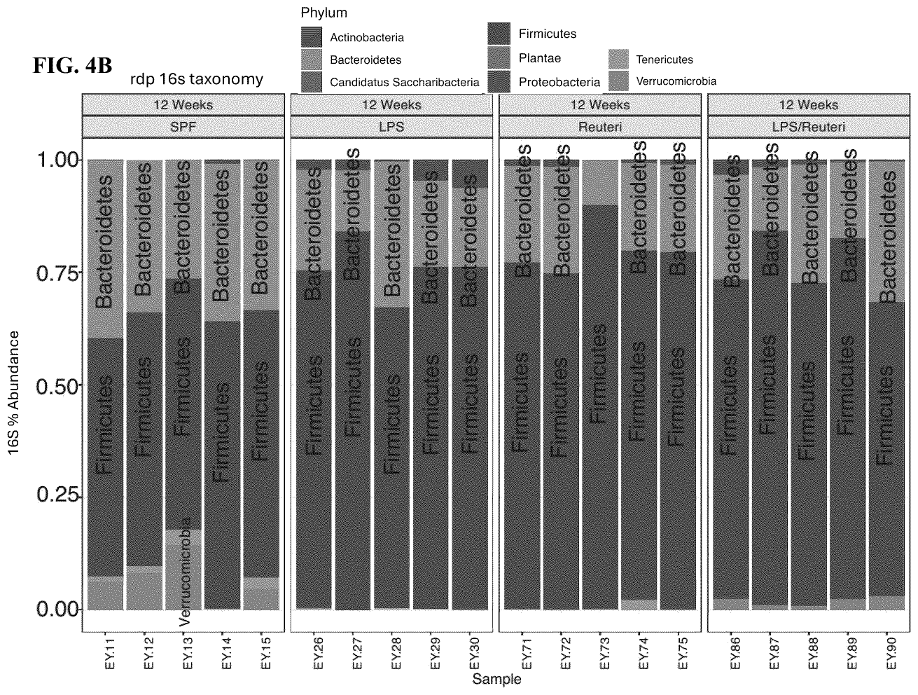

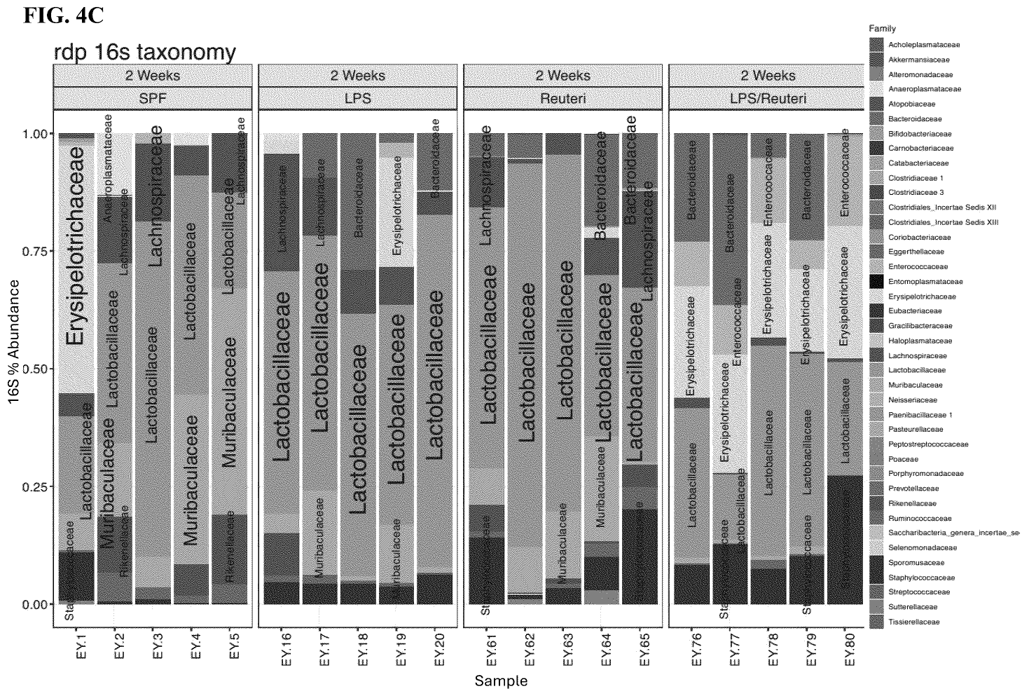

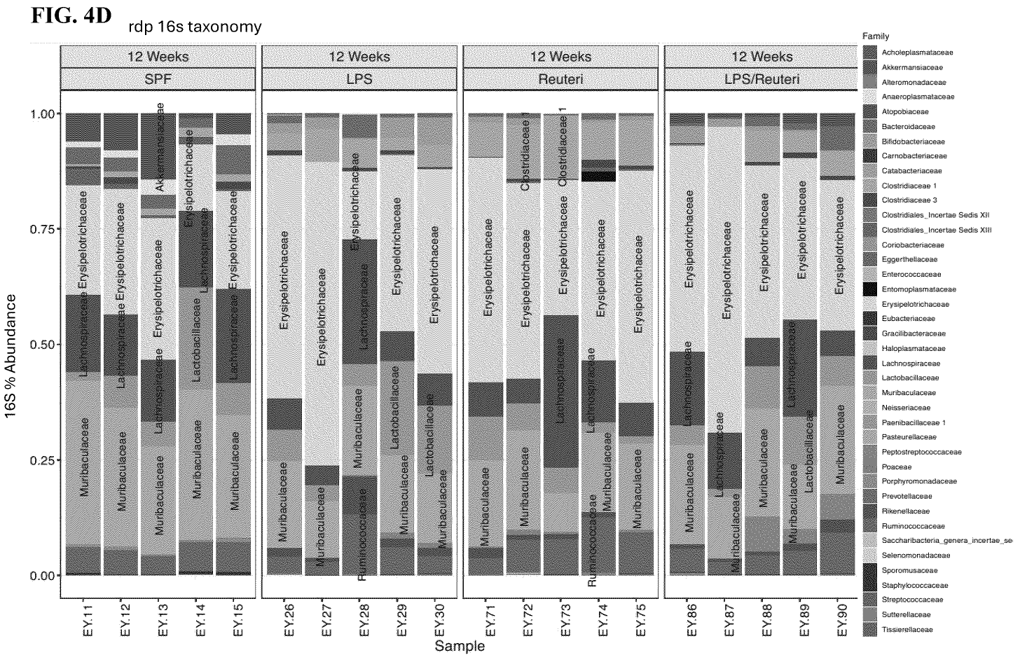

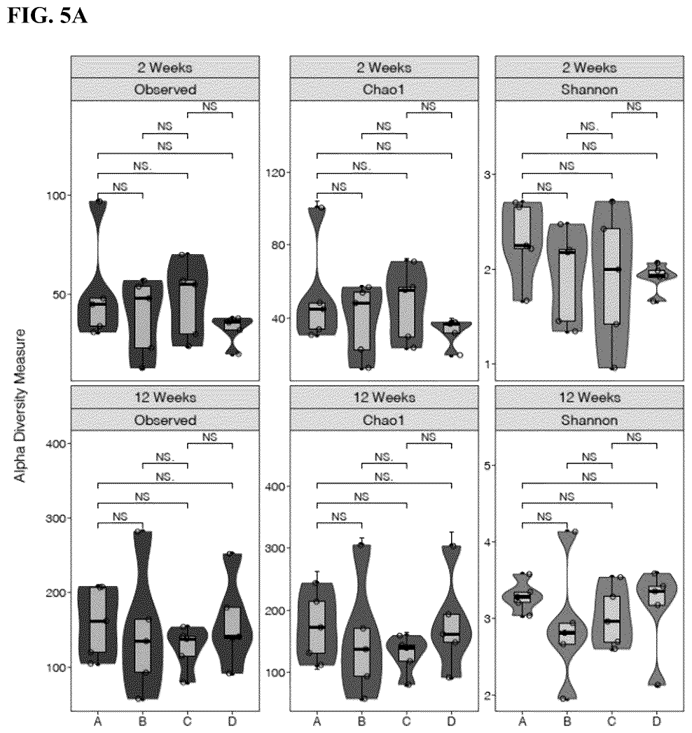

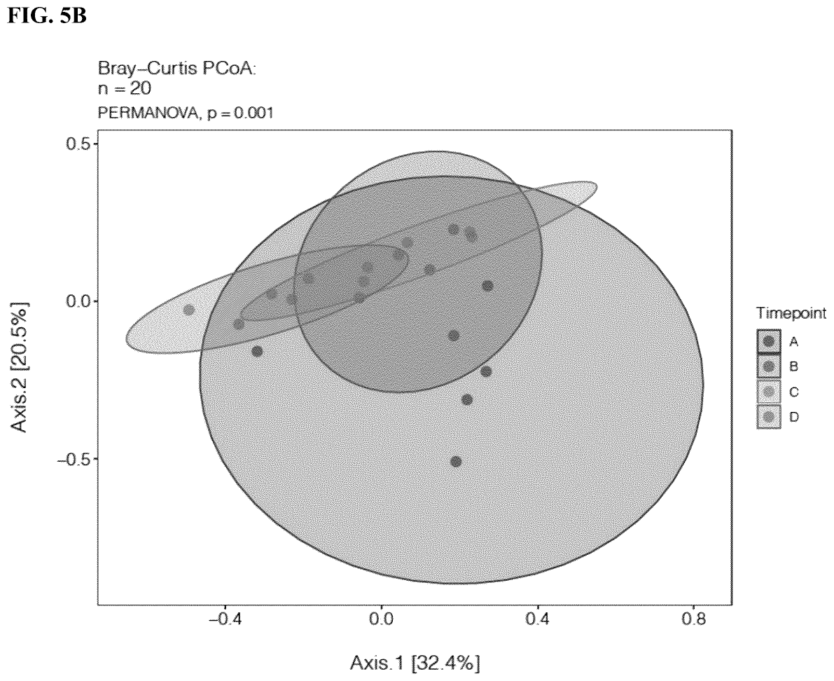

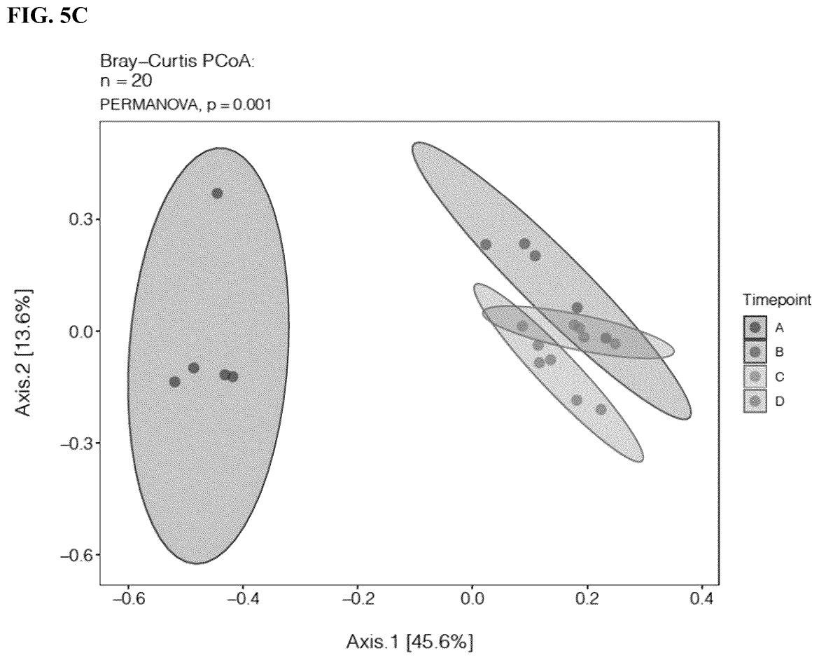

The impact of L. reuteri exposure on the offspring microbiome was next evaluated. Gut microbiome analysis based on relative abundance demonstrated no significant differences in taxa at the phylum or family levels among the four treatment groups (FIGS. 4a and 4b, all groups n=5). There was also no difference in α-diversity indices at two and 12 (FIG. 5a) weeks of age with either maternal LPS or L. reuteri supplementation during lactation. In contrast, the β-diversity of the gut microbiome among different groups showed differences as reflected in the principal coordinate analysis plot (PCoA) with Bray-Curtis dissimilarity at both two (FIG. 5b) and 12 weeks of age (FIG. 5c). PERMANOVA analysis revealed that there was a distinction in β diversity among treatment groups at both ages (both p=0.001). Taken together, these data demonstrate that maternal LPS exposure and/or L. reuteri supplementation during lactation modulates the β diversity of the gut microbiome.

Maternal Administration of L. reuteri During Lactation Reshaped Metabolomic Profile Shifts Induced by Maternal LPS Exposure

To test whether the bacterial composition changes observed based on β-diversity are associated with altered metabolic features, metabolic profiling was performed using serum and brain samples. This non-targeted metabolomics experiment resulted in 11,054 features. To reduce the information burden for interpretation of the data, features were computationally assigned putative molecular IDs using the GNPS online platform and only features that could be assigned an analogous match to fragmentation spectra found in the library were included in the analysis. Future feature list filtering as described in the Methods resulted in 389 high quality features with putative IDs. To determine if overall metabolic profiles were affected by the different treatment groups, a principal component analysis was used to perform unsupervised clustering of based on feature abundances.

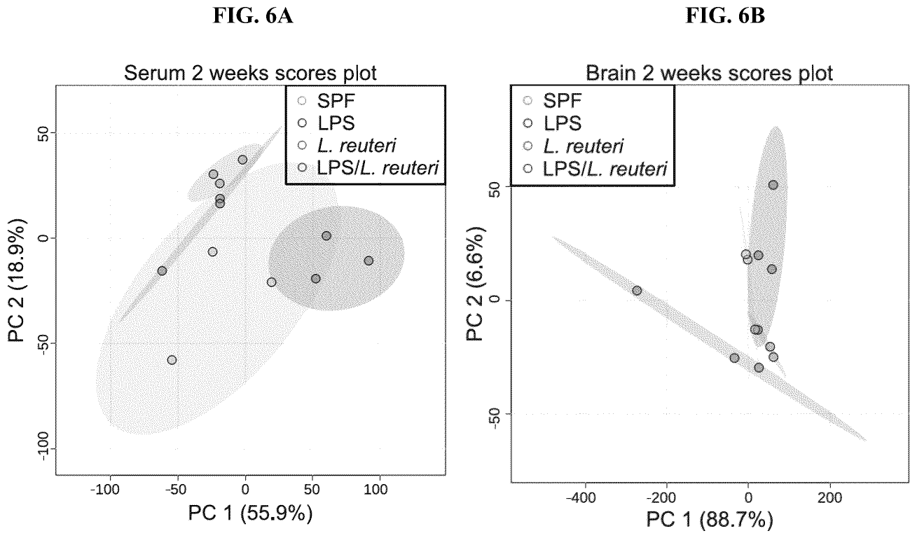

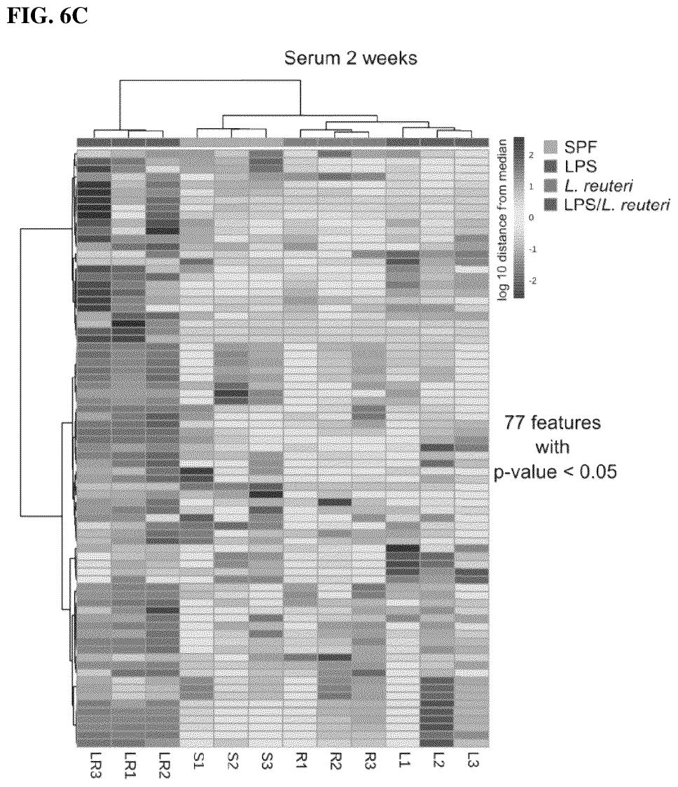

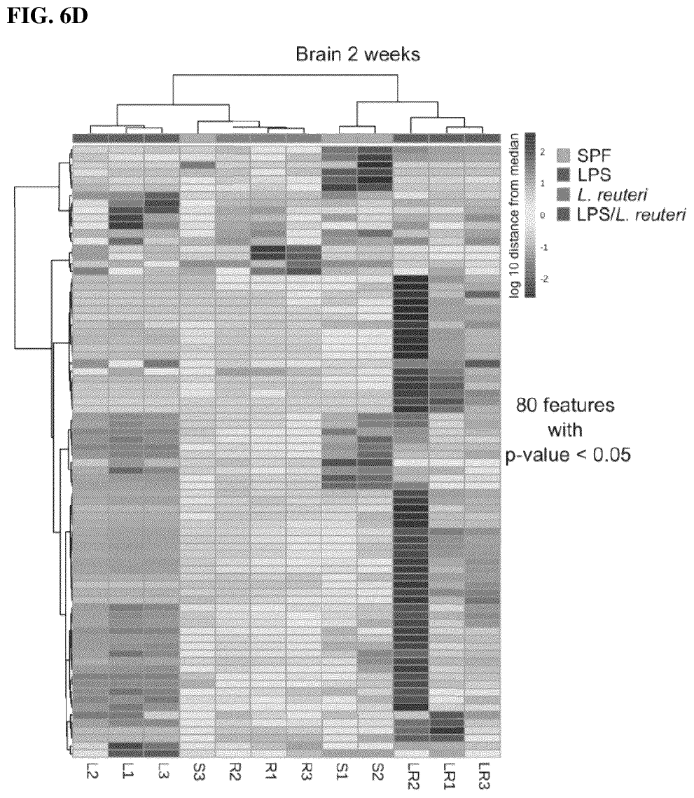

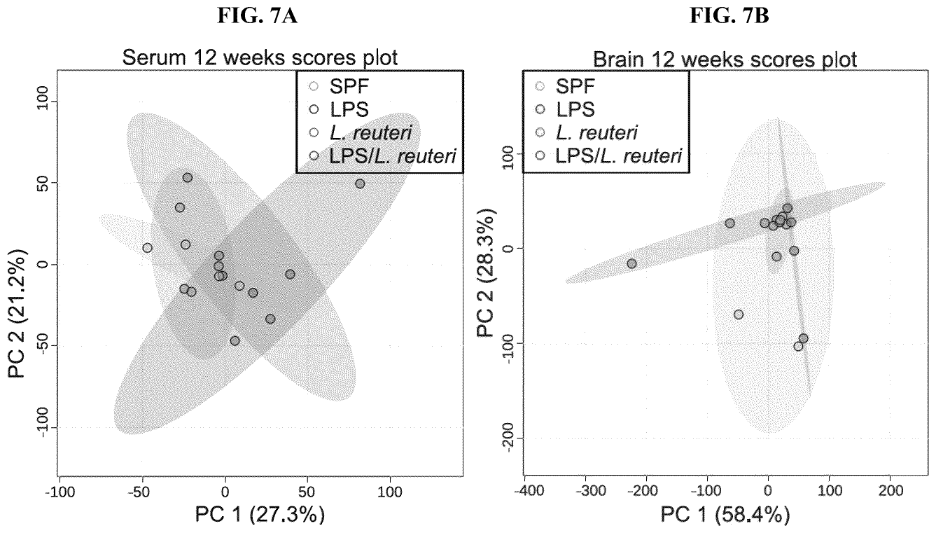

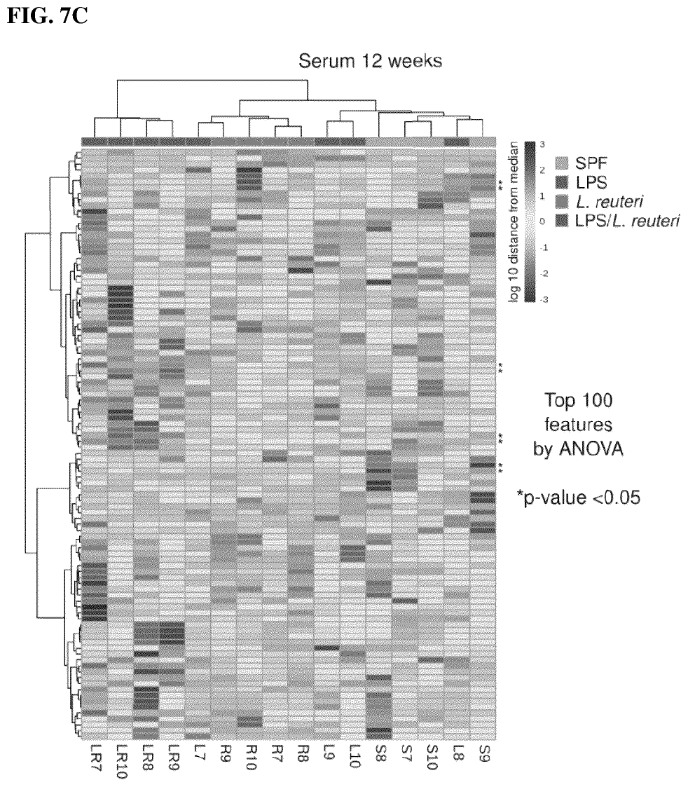

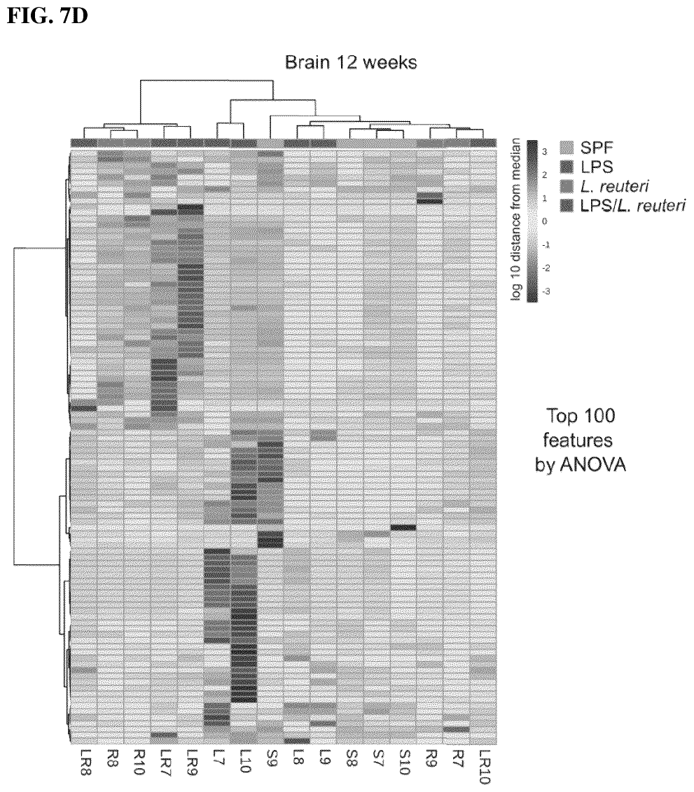

At two weeks old, metabolic profiles in both serum (FIG. 6a, all n=3) and brain (FIG. 6b, all n=3) samples clustered separately among the four treatment groups, with the clearest distinction in brain metabolic profiles observed in the LPS group compared to the other three groups. The distinct compositional clustering by treatments at two weeks of age is further demonstrated in the heatmaps of significantly differential abundance of the features shown in FIG. 6c, (serum with 77 features annotated) and FIG. 6d, (brain with 80 features annotated) based on one-way ANOVA with FDR (cutoff at 0.05)-adjusted p value. At 12 weeks of age, there was no separation in either the serum (FIG. 7a, all n=4) nor the brain (FIG. 7b, all n=4) metabolic pools. Using the same approach, at 12 weeks of age, the abundance of eight features in the serum (FIG. 7c) and no feature in the brain (FIG. 7d) were significantly different among the treatment groups as shown in the heatmaps.

Out of the 77 serum and 80 brain significantly differential features from two-weeks old mice, 20 were present in both pools, 60 were only in the brain pool and 57 features were only in the serum pool (FIG. 8a). Next, features associated with LPS that were altered by L. reuteri were specifically identified. Of the 20 features present in both pools, the levels of a lysophosphatidylcholine feature putatively identified as 1-palmitoyl-phosphatidylcholine (LysoPC(16:0)) were significantly decreased in the brains of LPS treatment group (p=0.0016) and restored by L. reuteri exposure (FIG. 8b, p<0.0001). Of the 60 features only present in the brain, the levels of two features putatively identified as LysoPC (20:5) and palmitoylcarnitine levels were reduced in the offspring brains of the maternal LPS-treated group but reestablished by L. reuteri exposure during lactation to levels similar to the SPF controls (FIG. 8c, for LysoPC (20:5), SPF vs LPS, p=0.0003, LPS vs LPS/Reuteri, p=0.0009; for palmitoylcarnitine, SPF vs LPS, p=0.0197, LPS vs LPS/Reuteri, p=0.0009). In the serum only pool, L. reuteri during lactation repaired maternal LPS-induced lower levels of a phosphatidylcholine feature annotated as of 1-(1Z-Hexadecenyl)-sn-glycero-3-phosphocholine (LPS vs LPS/Reuteri, p=0.0042) and reduced the maternal LPS-induced higher levels of a phosphatidylcholine feature putatively identified as PC(P-18:0/22:6) (FIG. 8d, LPS vs LPS/Reuteri, p=0.0066). Of the eight features identified with overall significant different abundance in the 12-week serum pool, no features associated with LPS were altered by L. reuteri.

To determine whether maternal LPS exposure induces new metabolic features that cross the BBB, a Venn diagram was used to sort features that were in the serum only pool in the untreated SPF but that appeared specifically in the brain of LPS-treated group (those also present in the LPS serum pool were excluded to focus investigation on those that newly crossed the BBB in response to LPS, FIG. 8e). An eicosanoid putatively identified as 8-hydroxyeicosatetraenoic acid (8-HETE) and a lysophosphatidylcholine feature putatively identified as 2-arachidonoyl-lysophosphatidylcholine, both derivatives of arachidonic acid, met these criteria. The relative abundance of these features was not significantly different among the treatment groups in the serum alone (data not shown) or brain alone pools of metabolites (FIG. 8f).

L. reuteri supplementation alone during lactation resulted in 14 new metabolites that crossed the BBB to the brain when compared to the untreated SPF controls (FIG. 8g). The majority of these metabolites with assigned putative IDs were lipid and lipid-like molecules that were bile acid or alcohol derivatives. Interestingly, none of these 14 metabolites were present in the SPF or LPS brain pools. Inspection of raw data revealed that these 14 features fell below the chosen noise and peak shape cutoffs for the curated data set in the brains of SPF and LPS mice suggesting that they were increased in the brains of L. reuteri exposed mice as a result of L. reuteri-associated metabolism.

### DISCUSSION

This study demonstrates that gestational MIA disrupts vascular formation and permeability early in life (before weaning), promotes astrocyte overactivation and results in behavioral alterations in spatial learning later in life. L. reuteri, an indigenous member of the human gut microbiome, when administered to lactating dams was shown to be an effective mean of rescuing the developmental deficits in the BBB and long-term cognitive function in their offspring. These findings were furthered supported by early changes (at two weeks old) in both the serum and brain metabolite profiles induced by gestational MIA and L. reuteri exposure during lactation. Specifically, metabolic products of lipid/bile acid metabolism associated with L. reuteri exposure during lactation can cross the BBB and may represent targets with potential beneficial bioactivities to remediate MIA insults. Notably, metabolic profiles at 12 weeks old, the time of observed differential cognitive function, were largely not affected by MIA or L. reuteri early life exposure. These findings suggest that there is an early developmental window key to optimization of the BBB through probiotics, and that this early optimization has long term impact on neurological outcomes later in life. In addition, administration of L. reuteri to the mother during lactation indicates that the beneficial effects of probiotics can be attained without directly exposing newborns to the probiotics, thus avoiding undesirable and potentially dangerous side effects of direct administration to newborns. Accordingly, the data presented herein indicates that probiotics can be used as a therapeutic agent to improve offspring outcomes when adverse conditions associated with MIA or chorioamnionitis is diagnosed. Together, these findings point to early BBB vasculature development as important for long-term cognitive function and as an early biomarker of gut-brain axis function.

This study further demonstrates that MIA (LPS) is a potent inducer of altered BBB development and function and CNS dysfunction. At two weeks of life rodent age, a stage of brain development equivalent to 22-37 weeks of gestation in humans, increased permeability of the BBB was observed, along with a significant BBB developmental deficit shown by the reduced total blood vessel volume.

In this study, pregnant SPF mice were given L. reuteri daily from birth to weaning (P21). Given that the process of the ensheathment of endothelial cells with astroglial end-feet occurs postnatally and BBB disruption is highly associated with reactive gliosis, the data presented herein demonstrates that maternal L. reuteri supplementation immediately after birth can specifically target astrocytes at both BBB and global levels in addition to normalizing MIA-induced developmental deficits in vessel development and increased BBB permeability.

To investigate the mechanism by which an altered microbiome may impact the brain, metabolic responses to MIA and L. reuteri on both vascular and brain sides of the BBB were studied. The eicosanoid annotated as 8-hydroxyeicosatetraenoic acid (8-HETE) and a lysophosphatidylcholine annotated as 2-arachidonoyl-lysophosphatidylcholine were shown herein to be able to cross the BBB of the offspring after maternal LPS challenge. Out of the 20 differentially present metabolites in both serum and brain pools, decreased level of a lysophosphatidylcholine annotated as Lyso PC (16:0) in the brain associated with maternal LPS was restored by L. reuteri exposure. The importance of microbiome associated metabolic features on the brain may not just be what crosses to the brain, but also what impacts BBB development directly. In the serum where potentially metabolites would have direct impact on BBB from the vascular side, L. reuteri modulated maternal LPS-induced decreased levels of a lysophosphatidylcholine annotated as 1-Hexadecyl-sn-glycero-3-phosphocholine and increased levels of another lysophosphatidylcholine annotated as PC(P-18:0/22:6) to levels similar to the controls.

L. reuteri exposure itself resulted in 14 additional metabolites that crossed the BBB. These metabolites are mostly related to lipid/bile acid metabolism. Bile acids regulate host glucose and lipid metabolism and absorption of fat and fat-soluble vitamins. Bile acids and metabolic products of bile acids have also been implicated in the communication between the gut microbiota and brain. Both primary bile acid and conjugated bile acids can cross the BBB but with different mechanisms. Primary bile acids cross the BBB through diffusion while conjugated bile acids utilize yet unidentified transporters on the BBB. The molecular role of bile acids in the CNS is largely unknown, however, different bile acid byproducts can have different effects on BBB function. The primary bile acids chenodeoxycholic acid and deoxycholic acid in the circulation can disrupt tight junctions and increase permeability of the BBB. Ursodeoxycholic acid (UDCA) and its glycine-conjugated form GUDCA protect brain endothelial cells from apoptosis. Rodent taurine-conjugated UDCA reduces neuroinflammation through its brain TGR5 receptor and has been implicated in several neurodegenerative diseases. Therefore, without wishing to be bound by theory it is possible that the protective effects on the BBB development and improved spatial learning by L. reuteri might be due to L. reuteri-induced bile acid-related metabolism.

In conclusion, MIA induced a developmental deficit in offspring vasculature formation associated with disrupted BBB integrity. BBB development deficiency early in life was associated with long-lasting effects on cognitive function. By introducing L. reuteri as an early microbial intervention during lactation, BBB development and cognitive function was improved. Regulation of metabolic responses to MIA through L. reuteri at both the vascular and brain sides of the BBB provide a potential mechanism and targets for promoting BBB integrity and long-term neurological outcomes.

### Methods

### Animals

Animal care and experimental procedures were approved by The University of Chicago Institutional Animal Care and Use Committee strictly in accordance with all guidelines by the U.S. National Institutes of Health. Timed pregnant C57BL/6J mice were kept on a 12-hour light/dark cycle and had access to food and water ad libitum. At gestational day 16 (E16), dams were randomized for injections of equivalent volumes (200 μL) of intraperitoneal (i.p.) LPS from Escherichia coli O55:B5 (Sigma-Aldrich, St. Louis, MO, USA) (50 ug/kg body weight of dam) or saline. Lactobacillus reuteri (ATCC PTA 6475) (L. reuteri) was cultured in an anaerobic cabinet (10% CO₂, 5% H₂, and 85% N2) overnight at 37° C. in MRS broth. Bacteria were spun down and suspended in an equivalent volume of serum-free DMEM medium. Right after delivery, both vehicle and LPS-challenged dams were further randomized to be fed daily (orally gavaged in a volume of 100 μL) with 10⁹ L. reuteri or vehicle until weaning or time points before weaning when pups were sacrificed for tissue collection or transferred to MRI. Another subset of pups was allowed to grow to the age of 12 weeks and subjected to behavioral testing. This resulted in four study groups: control, LPS, L. reuteri, LPS/L. reuteri.

### Morris Water Maze

Morris water maze was used to evaluate spatial learning and memory. Animal movements were registered and processed with ANY-maze software (Stoelting Co., Wood Dale, IL). Animal number in each treatment group is SPF (n=26, 13 females and 13 males from six litters), LPS (n=11, three females and eight males from three litters), Reuteri (n=8, two females and six males from three litters), and LPS/Reuteri (n=7, three females and four males from two litters). Briefly, mice were placed in a circular 120 cm diameter tank with room temperature (22° C.) water. At the training stage of the test, the mice were trained to locate a visible 10 cm diameter platform exposed 1 cm above the water. Five trials were performed and the platform location was changed for each trial. At the testing stage, mice were allowed to find the hidden platform that was submerged 1 cm below the surface in the southeast quadrant. Mice were tested for four consecutive days. On all testing days each mouse was subjected to five trials, each with a different starting position. The latency required to locate the platform (test duration, no more than 60 s) was recorded. The probe trial was performed on the 5th day, in which mice swam for 60 seconds with no platform in the tank. Time spent in the quadrant where the submerged platform had been in previous stages was recorded.

### MRI Experiments

### Protocol

Animals were anesthetized prior to imaging experiments, and anesthesia was maintained during imaging with 1.5-2.5% isoflurane. Temperature, heart and respiration rates were monitored and kept within normal range with a fiber optic detection system from SA Instruments (Stony Brook, NY, USA), designed for use in small animals. Animal number in each treatment group is SPF (n=5, two females and three males from two litters), LPS (n=8, five females and three males from four litters), Reuteri (n=7, four females and three males from three litters), and LPS/Reuteri (n=6, two females and four males from two litters).

MRI data were acquired on a 9.4 Tesla small animal scanner (Bruker, Ettlingen, Germany) with 11.6 cm inner diameter actively shielded gradient coils (maximum constant gradient strength for all axes—230 mT/m). Each mouse was placed supine on an animal holder and inserted into a 30 mm diameter quadrature volume coil (Rapid MR International, Columbus, OH). To cover the whole brain, multi-slice spin echo T2-weighted (T₂W) imaging was acquired along the coronal direction with a RARE (Rapid Acquisition with Relaxation Enhancement) pulse sequence (repetition time (TR)=4000 ms, echo time (TE)_{effective}=24 ms, field-of-view (FOV)=25.6×19.2 mm², matrix size=256×192, slice thickness=0.5 mm, RARE factor=8, number of excitations (NEX)=4). For the same geometry as T₂W imaging, time-of-flight (TOF) angiographic images were acquired with a flow compensated T₁-weighted sequence (TR/TE=15/3.9 ms, flip angle=60°, FOV=25.6×19.2 mm², 256×192, slice thickness=0.5 mm). Native T1 measurement was performed using RARE VTR (variable TR) images (TR=281, 350, 500, 1000, 1500, 2000, 3000, 5000, 10000 ms, TE=12.3 ms, RARE factor=4, FOV=25.6×19.2 mm², matrix size=128×96, thickness=1.5 mm, number of slice=9, NEX=1). 15 minutes after IP injection of 0.1 mmol/kg of Omniscan (gadodiamide, GE Healthcare, USA), the same T1 measurement as above was repeated twice.

### Data Analysis

MRI data were analyzed using IDL 6.4 (Harris Geospatial Solutions, Inc. CO, USA) with an in-house software package. The brain region-of-interest (ROI) was manually traced onto the T₂W image and superimposed on the TOF imaging and T1 measurement imaging. The whole brain volume was calculated by the sum of all pixels in each slice and multiplied by the slice thickness. To determine the volumes of blood vessels inside the brain, thresholds were set to select only for those pixels representing blood vessels. Using these pixels, the total volumes of blood vessels were calculated.

### Calculation of T₁

T₁ maps before and after contrast agent injection were calculated by fitting RARE VTR signal intensity (S_{TR}) in each pixel as a function of TR as follows:

$${S}_{T R} = {P}_{0} \cdot ( 1 - {e}^{- TR / {T}_{1}} )$$

where P₀ is the equilibrium signal depending on the proton density.

### Immunohistochemistry

Brains were freshly obtained from mice at postnatal age of two weeks and embedded and frozen in OCT. Eight m sections were fixed in ice-cold methanol for 20 minutes at −20° C. The samples were permeabilized with PBST with for 15 mins and then incubated with blocking solution (5% goat serum) in 0.2% Triton-X PBS (PBST) for one hour at room temperature (RT). The brain sections were then incubated with respective 50 μL of primary antibody solution overnight at 4° C. After wash with PBST four times for 10 mins, the sections were incubated with respective fluorophore-conjugated secondary antibodies for one hour at RT. DAPI-antifade mounting medium was used to counterstain nuclei (Invitrogen Inc., Carlsbad, CA, USA). Images were captured with a Stellaris confocal microscope (Leica Microsystems, Inc., Buffalo Grove, IL, USA). ImageJ (U. S. National Institutes of Health, Bethesda, Maryland, USA, http://imagej.nih.gov/ij/, 1997-2012)113 was used for imaging processing and analysis.

16S rRNA Sequencing

Mouse fecal samples were submitted to the Microbiome Metagenomics Facility of the Duchossois Family Institute (DFI) at the University of Chicago (Chicago, IL, USA) for genomic DNA extraction and subsequent 16S rRNA gene sequencing on the Illumina MiSeq platform. Dada2 (v1.18.0) as a default pipeline was used for processing MiSeq 16S rRNA reads with minor modifications in R (v4.0.3). Specifically, reads were first trimmed at 210 bp for forward reads and 150 for reverse reads to remove low quality nucleotides. Chimeras were detected and removed using the default consensus method in the dada2 pipeline. Then, ASVs with length between 300 bp and 360 bp were kept and deemed as high quality ASVs. Taxonomy of the resultant ASVs were assigned to the genus level using the RDP classifier (v2.13) with a minimum bootstrap confidence score of 80. Species-level classification can be provided using blastn (v2.13.0) and refseq_rna database (updated 2022 Jun. 10). Sequencing data was registered with NCBI Bioproject ID: PRJNA866398. At two weeks of age, animal number in each treatment group is SPF (n=5, two females and three males from two litters), LPS (n=5, two females and three males from two litters), Reuteri (n=5, four females and one male from two litters), and LPS/Reuteri (n=5, two females and three males from two litters). At 12 weeks of age, animal number in each treatment group is SPF (n=5, one female and four males from two litters), LPS (n=5, three females and two males from two litters), Reuteri (n=5, one female and four males from two litters), and LPS/Reuteri (n=5, three females and two males from two litters).

### Metabolomic Analysis

### Data Collection

Serum and brain samples were submitted to the Microbiome Metagenomics Facility of the DFI for metabolite extraction. Samples were analyzed on a Thermo Fisher liquid chromatography system coupled to an Orbitrap IQ-X mass spectrometer, operating in positive mode. 3 μL of sample was injected onto a Cortecs© UPLC T3 Column (1.2 μm, 2.1×100 mm) fitted with Cortecs© UPLC T3 guard at 30° C. The mobile phase A was water with 0.1% Formic Acid and mobile phase B was 95% Acetonitrile with 0.1% Formic Acid. Gradient elution started with 0% B with a flow rate of 0.48 mL/min for 0.2 min and linearly increased to 97% B over 5 min and these conditions were held constant for 1.0 min. Finally, re-equilibration at 0% B was performed for 1.5 min. The electrospray ionization conditions were set with the spray voltage at 3.4 kV, vaporizer temp at 400° C., and detection window set to 100-2000 m/z. Precursor selection for MS² scans was set to 150-2000 m/z with dynamic exclusion after 2 times within 10 seconds. The isolation window was 1.5 m/z with no offset and a fixed collision energy of 30%. At two weeks of age, animal number in each treatment group for serum samples is SPF (n=3, two females and one male from three litters), LPS (n=3, two females and one male from two litters), Reuteri (n=3, two females and one male from two litters), and LPS/Reuteri (n=3, two females and one male from one litter). At 12 weeks of age for serum samples, animal number in each treatment group is SPF (n=4, two females and two males from two litters), LPS (n=4, two females and two males from three litters), Reuteri (n=4, two females and two males from two litters), and LPS/Reuteri (n=4, two females and two males from two litters). At two weeks of age, animal number in each treatment group for brain samples is SPF (n=3, one female and two males from two litters), LPS (n=3, three females from two litters), Reuteri (n=3, two females and one male from two litters), and LPS/Reuteri (n=3, one female and two males from one litter). At 12 weeks of age for brain samples, animal number in each treatment group is SPF (n=4, two females and two males from two litters), LPS (n=4, two females and two males from three litters), Reuteri (n=4, two females and two males from two litters), and LPS/Reuteri (n=4, two females and two males from two litters).

### Data Processing

Raw data files were converted into open source file format and processed using MZmine2 and the Feature-Based Molecular Networking function in the Global Natural Products Social Molecular Networking (GNPS) environment to identify features and match data to publicly available library spectra. MetaboAnalyst was used for statistical analysis and visualizations.

### MZmine

MZmine 2.53 (Pluskal, T., et al., MZmine 2: modular framework for processing, visualizing, and analyzing mass spectrometry-based molecular profile data. BMC Bioinformatics 11, 395, doi:10.1186/1471-2105-11-395 (2010)) was used to create feature lists with abundances in each sample from the raw data. Settings used were based on manual inspection of the raw data for values that represented signals above the inherent noise level, typical peak shapes, and mass and retention time (RT) tolerances. First, a mass detection was used with a noise cutoff filter for both MS1 and MS2 scans to create a mass list for each data file. The Centroid mass detector was set to a level of 6.0E3 for the MS2 level and 1.0E4 for the MS1 level. The ADAP chromatogram builder algorithm was used to create extracted ion chromatograms at the MS1 level with a minimum group size of 3 scans, group intensity threshold of 1.0E4, minimum highest intensity of 3.0E4, and m/z tolerance of 0.015 Da or 5.0 ppm. Chromatogram deconvolution was performed using the Wavelets (ADAP) algorithm with a S/N threshold of 10, minimum feature height of 5E5, coefficient/area threshold 110, Peak duration range of 0.00-1.00, RT wavelet range of 0.01-0.25, median m/z center calculation, m/z range for MS2 scan pairing 0.01 Da, RT range for MS2 scan pairing 0.1 min. The isotopic peak grouper module was used to group features that are isotopes with m/z tolerance of 0.01 Da or 5.0 ppm, RT 0.1 min, a maximum charge of 4, and the lowest m/z as representative isotope. A master feature list was created using the Join aligner module with m/z tolerance of 0.01 Da or 5.0 ppm, RT 0.1 min, and weight for m/z and RT set to be equal (1). The resulting feature list was filtered to remove duplicate features and then the gap-filling algorithm was used to fill in any missing values for peaks that were not detected with the previous algorithms. The peak-finder gap filling algorithm was used with intensity tolerance 10%, m/z tolerance 0.02 Da or 5.0 ppm, and RT tolerance 0.1 min. Peaks were filtered to remove any with a peak area less than 3.0E4. The resulting feature list was exported for GNPS analysis.

### The Global Natural Product Social Molecular Networking (GNPS)

A molecular network was created with the Feature-Based Molecular Networking (FBMN) workflow (Nothias, L. F. et al. Feature-based molecular networking in the GNPS analysis environment. Nat Methods 17, 905-908, doi:10.1038/s41592-020-0933-6 (2020)) on GNPS (gnps.ucsd.edu). The results from MZmine2 were exported to GNPS for FBMN analysis. The data was filtered by removing all MS/MS fragment ions within +/−17 Da of the precursor m/z. MS/MS spectra were window filtered by choosing only the top 6 fragment ions in the +/−50 Da window throughout the spectrum. The precursor ion mass tolerance was set to 0.02 Da and the MS/MS fragment ion tolerance to 0.02 Da. A molecular network was then created where edges were filtered to have a cosine score above 0.7 and more than 6 matched peaks. Further, edges between two nodes were kept in the network if and only if each of the nodes appeared in each other respective top 10 most similar nodes. Finally, the maximum size of a molecular family was set to 100, and the lowest scoring edges were removed from molecular families until the molecular family size was below this threshold. The analogue search mode was used by searching against MS/MS spectra with a maximum difference of 100.0 in the precursor ion value. The library spectra were filtered in the same manner as the input data. All matches kept between network spectra and library spectra were required to have a score above 0.7 and at least 6 matched peaks. The job can be accessed at gnps.ucsd.edu/ProteoSAFe/status.jsp?task=95ff61735c414baabd25a8fc0aeb7888.

### Feature List Filtering

The feature list from GNPS was exported with putative IDs associated and filtered in Excel using peak areas found in blanks and quality control injections to remove low quality features from the statistical analysis. To filter out blank peaks, first any features with raw peak area of over 1E6 found in any solvent blank sample were removed. Features within a sample were then normalized by dividing peak area by the peak area of the cholic acid internal standard in that sample. Features with normalized peak areas in samples less than or equal to the peak areas found in method blank controls were removed. A pooled QC of each sample treatment group was injected throughout the run and 3 injections of each QC were used to calculate the percent coefficient of variation (% CV) of features. Any features with normalized peak area % CV of greater than 10% across the 3 pooled QC injections was eliminated. Finally, features that GNPS matched known mass spectrometry contaminants and polyether polymers were removed, resulting in a list of 389 features.

### MetaboAnalyst

MetaboAnalyst (Pang, Z. et al. MetaboAnalyst 5.0: narrowing the gap between raw spectra and functional insights. Nucleic Acids Res 49, W388-W396 (2021)) was used to statistically analyze and visualize the feature list exported from GNPS and filtered down to 389 high quality features. For each subset, any features that had a single or constant value across all samples were removed. Figures were exported from MetaboAnalyst.

### Statistics

16S rRNA sequencing and metabolomics data analysis are stated in their respective sections above. All other data are presented as mean±standard error of the mean (SEM). GraphPad's Prism 9 (La Jolla, CA) software was used to perform statistical analyses. One-way ANOVA with Tukey's multiple comparisons post hoc test was used to determine differences among multiple groups. A p-value of <0.05 was considered significant.

1. A method of treating one or more neurodevelopmental deficits in a subject, the method comprising providing one or more probiotics to the subject via indirect maternal administration.

2. The method of claim 1, wherein the one or more probiotics comprise Limosilactobacillus reuteri.

3. The method of claim 1, wherein the subject was exposed to maternal immune activation (MIA) in utero.

4. The method of claim 3, wherein the subject was exposed to MIA after at least 20 weeks of gestation.

5. The method of claim 4, wherein the subject was exposed to MIA after at least 27 weeks of gestation.

6. The method of claim 3, wherein the subject was exposed to MIA as a result of an autoimmune condition, asthma, an allergic condition, a viral infection, and/or a bacterial infection in the mother during gestation of the subject.

7. The method of claim 1, wherein the subject was exposed to MIA as a result of chorioamnionitis during gestation of the subject.

8. The method of claim 1, wherein the one or more neurodevelopmental deficits are selected from blood brain barrier (BBB) dysfunction, neuronal apoptosis, hypomyelination of white matter, neuroinflammation, microgliosis, astrogliosis, periventricular leukomalacia, neonatal encephalopathy, cerebral palsy, neonatal stroke, cognitive impairment, language impairment, autism, and schizophrenia.

9. The method of claim 1, wherein the one or more probiotics are provided to the subject via indirect maternal administration during gestation of the subject.

10. The method of claim 1, wherein the one or more probiotics are provided to the subject via indirect maternal administration after the subject is born.

11. The method of claim 1, wherein the one or more probiotics are provided to the subject via indirect maternal administration during gestation of the subject and via indirect maternal administration after the subject is born.

12. The method of claim 1, wherein the dose of probiotics provided to the mother to achieve indirect maternal administration of the probiotics to the subject is at least about 10 million colony forming units (CFU).

13. The method of claim 1, wherein the one or more probiotics modulate the microbiome and/or metabolome of the mother and/or the subject.

14. The method of claim 13, wherein the one or more probiotics modulate production of metabolites in the mother, and metabolites produced by the mother cross the blood brain barrier of the subject.

15. The method of claim 1, wherein the subject is a human.
